(19) United States
(12) Patent Application Publication
(10) Pub. No.: US 2024/0065999 A1
(43) Pub. Date: Feb. 29, 2024

(54) Ophthalmic Formulation Containing a Dopaminergic Prodrug that may be Combined with One or More Agents

(71) Applicant: The Board of Trustees of the Leland Stanford Junior University, Stanford, CA (US)

(72) Inventors: Ira Schachar, Stanford, CA (US); Cassie Ludwig, Stanford, CA (US); Stephen J. Smith, Stanford, CA (US)

(21) Appl. No.: 18/023,839

(22) PCT Filed: Nov. 9, 2021
(86) PCT No.: PCT/US2021/058624

§ 371 Date: Feb. 28, 2023

Related U.S. Application Data
(60) Provisional application No. 63/111,216, filed on Nov. 9, 2020.

(51) Int. Cl. A61K 31/223; A61K 9/00; A61K 9/08; A61K 31/137; A61P 27/10

(52) U.S. Cl. CPC A61K 31/223; A61K 9/0048; A61K 9/08; A61K 31/137; A61P 27/10

(57) ABSTRACT

The invention described herein relates to a prodrug that is converted into a dopamine agonist. The dopamine prodrug may be combined with one or more dopamine antagonists, dopamine receptor inhibitors, or vesicular monoamine transport inhibitors. A prodrug is a biologically inactive compound that can be metabolized by the body into its active form. For example, a prodrug placed on the ocular surface can undergo hydrolysis during penetration of the ocular surface, resulting in a biologically active drug. A prodrug allows for use of a lower concentration of the drug and protects against undesirable side effects. A prodrug can also help augment drug uptake into the target tissue.

### CROSS-REFERENCE TO RELATED APPLICATIONS

This application is a U.S. National Stage Application of PCT Application No. PCT/US2021/058624 filed Nov. 9, 2021, which application, pursuant to 35 U.S.C. § 119 (e), claims priority to the filing date of U.S. Provisional Patent Application Ser. No. 63/111,216, filed Nov. 9, 2020, the disclosure of which applications are incorporated herein by reference.

### FIELD

The present invention relates to the field of a prodrug that is converted into a dopamine agonist that may be combined with one or more dopamine antagonists, dopamine reuptake inhibitors, or vesicular monoamine transport inhibitors.

### BACKGROUND

Myopia affects one third of the world's population today and is predicted to affect 50% of the world's population by 2050. In contrast with the apparent declining incidence and prevalence of both early and late macular degeneration there has been an increase in all forms of myopia both in the US and in Asia. A study by Chen and colleagues in 2018 found that the increase in high myopia (>-6D) and very high myopia (>-10D) contributed in large part to the increasing trend of myopia prevalence from 2001 to 2015 in eastern China. This increasing prevalence has significant consequences as high myopia can lead to myopic maculopathy, choroidal neovascularization, myopic macular degeneration, myopic traction maculopathy, retinal detachments, intrachoroidal cavitations, a dome shaped macula, strabismus, glaucoma, and an increase in the difficulty and risks of cataract surgery. Slowing myopia progression even minimally can help prevent blindness. Using combined data from five large population-based studies, Bullimore et al. found that slowing myopia by 1 diopter should reduce the likelihood of a patient developing myopic maculopathy by 40%.

The pathway of axial length homeostasis is as follows: light stimulates photoreceptors which signal ON-bipolar cells which activate dopaminergic amacrine cells to secrete dopamine which in turn stimulates glucagonergic amacrine cells. These signal to the retinal pigmented epithelium which signals to the choroid which signals to the sclera to change its structure. This pathway is tied to both the circadian pathway involving melatonin which is increased in the dark, while dopamine is increased in the light. It is also tied to acetylcholinergic amacrine cells which can inhibit dopaminergic amacrine cells and can themselves signal to the choroid.

Several mediators in the pathway have been examined in eloquent experiments of up and down regulation to determine whether manipulating these can also induce or inhibit myopia. These have, for example, included injecting insulin into chick eyes that received treatment with negative lenses to induce myopia development. In an experiment by Vogt et al. in 2009, this led to a 2-fold increase in intravitreal dopamine after three days of treatment. While much work is being done to look at the direct upstream and downstream roles of other amacrine cells, retinal pigment epithelium, choroid, and scleral signaling, it appears very clear that manipulation of dopamine alone can directly induce or inhibit myopia.

Experiments done in chicks, mice, guinea pigs, tree shrews, and macaques have demonstrated that injection of dopamine agonists blocks myopia while injecting dopamine antagonists is myopigenic. Similarly, injection of acetylcholine antagonists has been shown to inhibit myopia, while acetylcholine agonists induce myopia.

More recently, a cellular substrate for an image focus signal which controls emmetropization has been found. Schwartz et al. identified murine ON delayed retinal ganglion cells (OND RGCs) with receptive field properties differing from the typical center-surround organization of RGCs in 2017. They found that unlike canonical center-surround RGCs, OND RGCs respond earlier as a visual stimulus increases in size and can be excited by light falling far beyond their dendrites. The OND RGCs were believed to report a more global property of the visual scene—integrating long temporal and large spatial scales. Dopamine receptors exist on retinal neurons including RGCs, potentially explaining the regulatory role of dopamine in the emmetropization pathway.

Genetics also appear to play a role in myopia, with heritability thought to be between 60% and 80% according to the IMI white paper on Genetics in 2019. To date, nearly 200 genetic loci have been identified for refractive error and myopia. GWAS studies have added evidence for light-induced signaling as an important driver of refractive error. Two genes that have been consistently associated with myopia development are the RASGRF1 gene—which is linked to photoreceptor function and GJD2 which plays a role in coupling and uncoupling of rods and cones, horizontal cells and amacrine cells. It is regulated by phosphorylation which in turn is controlled by dopamine

There are two important animal models for myopia: form deprivation myopia and lens induced myopia. In 1977 Wiesel et al. sutured either one or both eyelids of ten macaques shut at various times after birth. They found an overall distension of the post equatorial hemisphere and attenuation of the posterior sclera in a macaque whose eyelids were sewn from 2 weeks through 18 months of age. In all such experiments, the degree of myopia increased with duration of occlusion, and increased with decreased age at time of occlusion. This type of occlusion is known as form deprivation myopia and is considered an open loop process as eyes have no way to overcome reduced spatial and temporal contrast, so continued growth does not reduce the level of stimulation towards growth. Therefore, eyes continue to grow until natural reductions in body growth terminates the process. By comparison, lens induced myopia, in which a negative lens is placed over the eye and the eye elongates until emmetropization is reached is a closed loop paradigm. This is a precise compensation to the lens. Lens induced myopia was first studied in chicks in 1988 and was shown by Wallman et al. in 2004 to be spatially and temporally dependent.

Light exposure and time spent outdoors have been negatively associated with risk of myopia in large epidemiologic studies as well as in animal models of myopia. In a study in 2010 in which Ashby et al. fitted chicks with translucent diffusers for 5 days and removed the diffusers for 15 minutes daily under one of three lighting conditions, chicks who had 15 minutes daily of 15,000 or 30,000 lux lighting had reduced levels of form deprivation myopia. However, this protective effect was abolished by the daily injection of spiperone, a dopamine antagonist acting on D2 receptors. Clinical trials found that increasing outdoor activity led to a 13% reduced odds for each additional hour of time spent outdoors each day. Current recommendations are that children spend at least 2 hours per day outdoors to prevent myopia.

The only drug currently in trials in the United States for use in inhibiting axial length elongation is atropine, a nonspecific muscarinic acetylcholine antagonist. There are five muscarinic receptors in the eye, M1-M5, and atropine's actions on M2 in the sclera have been implicated in its efficacy in inhibiting myopia. Atropine's efficacy was initially thought to be through inhibiting accommodation, but it has been found to work at doses as low as 0.01%, doses at which it does not strongly inhibit accommodation. Furthermore, Atropine was found to work to inhibit induction of myopia in chicks whose accommodation functions through nicotinic rather than muscarinic receptors. Its site of action was then initially thought to be in the retina, but in 1998, when Fischer and colleagues used a cholinergic toxin to destroy cholinergic amacrine cells in the retina of chicks, form deprivation myopia and lens induced myopia still developed and these could still be blocked with atropine.

A paper by McBrien et al. found no up-regulation of acetylcholine mRNA after myopia induction in guinea pigs, but a paper by Barathi et al in 2013 found that mutant mice lacking the M2 receptor were less likely to develop myopia. They found that these mice had reduced scleral fibroblast growth and a higher ratio of collagen type 1 as compared to type 5, demonstrating the likely downstream effects of atropine on the inhibition of myopic progression.

In the ATOM trial, 1% atropine instilled nightly in one eye over a two-year period reduced myopic progression by 77%—an approximate +0.8 diopter difference. Axial length remained unchanged in the atropine group but increased by 0.4+/−0.5 mm in placebo. However, there was a strong rebound phenomenon, raising concern about the long term benefits relative to the risks of atropine use (including but not limited to potential abnormalities of accommodation, photophobia, allergic or hypersensitivity reactions, glare, subjectively decreased visual acuity, dry eye, dry mouth, dry throat, flushed skin and constipation).

Bifocals, progressive additional lenses, glasses and contact lenses made to account for peripheral blur and Orthokeratology (Ortho-K) all likely work via a reduction in peripheral blur, and therefore via increased levels of dopamine release from dopaminergic amacrine cells. Their effects in preventing progression are unfortunately modest with an average approximate reduction in progression of 0.3D for progressive lenses, glasses and contact lenses and of 0.4D for Ortho-K in larger studies.

Researchers have also tried to address the most downstream aspect of axial elongation at the level of the sclera, so far without success. Scleral reinforcement surgery included using scleral grafts, had success in one large study by the inventor, but very mixed results in other studies and has been discredited. Scleral shortening has shown little improvement with refraction and is not currently in use. There is still poor data behind scleral strengthening with polymers injected into collagen. And scleral cross-linking is being examined, but its safety and efficacy have not yet been assessed.

Deprivation myopia has been effectively altered by intravitreal injection of drugs that affect retinal dopamine or serotonin levels, including reserpine, suggesting the modulation of dopamine levels via dopamine receptor inhibitors or vesicular monoamine transport inhibitors may also modulate axial elongation (Schaeffel et al, 1994).

In spite of the high prevalence of myopia, no pharmacologic treatment has been approved or cleared for myopia control by the U.S. Food and Drug Administration. This likely stems from the failure of current therapies to directly increase the central mediator in the pathway of axial length elongation:

dopamine Several patents have been filed for dopaminergic agents to treat myopia (U.S. Pat. No. 5,814,638A, CN-105434439-A, JP-2019505542-A, KR-0159640-B1, CN-105434439-A,W0-0054773-A1,US-2017020891-A1). The problem with using an isolated dopaminergic agent is that the side effects may lead to termination of use. Dopaminergic agents are known to cause nausea and vomiting—both Muller et al. in 1959 and Cote et al. in 2008 reported use of conjunctival apomorphine (a nonselective dopaminergic agent) to induce emesis in dogs who have consumed foreign bodies. Additionally, dopaminergic agents are known to cause conjunctivitis. In fact, 8 of 17 patients using ibopamine for hypotony stopped using the drug before 24 weeks due to follicular conjunctivitis or irritation (Ganteris-Gerritsen et al. 2012). Others have attempted to pair dopaminergic agents with antiallergic agents due to this known side effect (JP-2019505542-A).

### SUMMARY

Provided herein is a novel approach, using a dopaminergic prodrug so as to avoid negative effects of the dopaminergic agent on the conjunctiva while still allowing for penetration of the medication past the ocular surface. If necessary, the prodrug might also be combined with a dopaminergic antagonist so as to further block any systemic side effects. Herein we disclose novel pharmacologic compounds to increase ocular dopamine while preventing the known side effects of dopaminergic agents outside of the eye.

### DESCRIPTION OF THE DRAWINGS

FIG. 1 provides an overview of a dopamine prodrug combined with an agent to prevent D1 action while outside the eye, with drug hydrolysis upon passing through the ocular tissue resulting in D1 agonist action inside the eye. The example drug in this illustration is ibopamine

FIG. 2 provides an overview of a dopamine prodrug combined with an agent to prevent D1 action while outside the eye, with drug hydrolysis upon passing through the ocular tissue resulting in D1 agonist action inside the eye. The example drug in this illustration is adrogolide derivatives

FIG. 3 provides an overview of a dopamine prodrug combined with an agent to prevent D1 action while outside the eye, with drug hydrolysis upon passing through the ocular tissue resulting in D1 agonist action inside the eye. The example drug in this illustration is 3,4-Dibenzoyl dopamine derivatives

FIG. 4 provides an overview of a dopamine prodrug combined with an agent to prevent D1 action while outside the eye, with drug hydrolysis upon passing through the ocular tissue resulting in D1 agonist action inside the eye. The example drug in this illustration is docarpamine derivatives

FIG. 5 provides an overview of a dopamine prodrug combined with an agent to prevent D1 action while outside the eye, with drug hydrolysis upon passing through the ocular tissue resulting in D1 agonist action inside the eye. The example drug in this illustration is etilevodopa derivatives

FIG. 6 provides an overview of a dopamine prodrug combined with an agent to prevent D1 action while outside the eye, with drug hydrolysis upon passing through the ocular tissue resulting in D1 agonist action inside the eye. The example drug in this illustration is SK&F R-105058 derivatives

FIG. 7. Ex vivo vitreous penetration effect of dopamine and dopaminergic derivatives. Concentration of dopaminergic derivatives was quantified in the vitreous of porcine eye after a single topical treatment of 30 μL 0.5% dopamine, 0.5% levodopa, 0.5% etilevodopa, or 10% etilevodopa at 24 hours. All the studied drugs were able to significantly penetrate the eye and diffuse into vitreous. PBS was used as a control. All values represent the mean±SEM, and at least six porcine eyes were studied for each treatment. *P<0.05 relative to control (PBS) by unpaired t-test.

FIG. 8. Ex vivo pharmacokinetic profile of anterior chamber (Panel A) and vitreous (Panel B) penetration of etilevodopa 10% over 72 hours. Etilevodopa continuously diffused into the anterior chamber and vitreous of porcine eye from the ocular surface over a 72-hour period after a single administration of topical etilevodopa 10%. The results are graphed as a log-linear plot. All values represent the mean±SEM, and at least three porcine eyes were studied for each time point.

FIG. 9. In vivo pharmacokinetic effect of 24 hours (Panel A) and four weeks (Panel B) treatment of etilevodopa. (Panel A) Concentration of dopaminergic derivatives at 24 hours was quantified in whole Long Evans rat eye, retina/vitreous and serum after a single treatment of etilevodopa 10% as eye drop. The concentrations declined over 24 hours. The results are graphed as a log-linear plot. All values represent the mean±SEM, n≥3. (Panel B) Concentration of dopaminergic derivatives in the rats serum at 14 and 28 days after once-daily topical administration of etilevodopa 10%. All values represent the mean±SEM, and four rats were studied at each time point.

FIG. 10. Clinical, structural and functional assessment of Long Evans rat eyes after topical administration of etilevodopa. Etilevodopa 10% 5 μL was administered as an eye drop to one eye of each rat once daily, while the contralateral eye received PBS as control. Representative photos of a rat eye (Panel A) at days 14 and 28 show no evidence of inflammatory response, corneal opacity, fundus abnormalities, or conjunctival redness. Ocular coherence tomography (OCT) images of the retina (Panel B) demonstrate no qualitative changes in retinal structure at 14 or 28 days. (Panel C) Comparison of electroretinography (ERG) waveform of etilevodopa with PBS control treated eyes at 14 and 28 days. No statistically significant changes in the A-wave amplitude (Panel D) or B-wave amplitude (Panel E) were observed at studied time points. All values represent the mean±SEM and 4 rats were studied at each time point. Statistical comparisons were performed with two-way ANOVA followed by Sidak analysis.

FIG. 11. Hematoxylin and eosin staining of rat eyes 14 and 28 days after topical administration of 5 μL etilevodopa 10% or PBS once daily. There was no inflammatory or fibrotic response in either control or treatment groups at studied time points.

FIG. 12. Cleaved caspase-3 immunostaining analysis of rat eyes at 14 and 28 days after topical treatment of 5 μL etilevodopa 10% or PBS once daily. (Panel A) Cleaved caspase-3+ DAPI+ immunostaining was primarily observed in the inner nuclear layer of both PBS control group eyes and treatment group eyes at day 14 and 28 days. Scale bar: 50 μm. (Panel B) Bar graph shows no statistically significant change in the number of cleaved caspase-3+ DAPI+ cells between control and treatment groups at studied time points. All values represent the mean±SEM. Statistical comparisons were performed with two-way ANOVA followed by Sidak analysis.

FIG. 13. TUNEL immunostaining analysis of rat eyes at 14 and 28 days after topical treatment of 5 μL etilevodopa 10% or PBS once daily. There were no TUNEL+ DAPI+ cells observed in either control or treatment groups at studied time points. Scale bar: 50 μm.

### DEFINITIONS

FDM: form deprivation myopia
LIM: lens induced myopia
DA: dopamine
D1: dopamine receptor D1
D2: dopamine receptor D2
IOP: intraocular pressure

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular conjunctival tissue, nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

The term “sterile” is used in the conventional sense to denote free from live bacteria or other microorganisms. A “sterile field” is an area within the operating theater/clinic within which only sterile equipment can be used, and into which only those personnel who have gone through surgical scrubbing and the gowning process can enter.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as an antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In addition to the active agent or agents, a given active agent composition includes a pharmaceutically acceptable delivery vehicle, e.g., a pharmaceutically acceptable aqueous vehicle. For pharmaceutically acceptable aqueous vehicles, in addition to water, the aqueous delivery vehicle may include a number of different components, including but not limited to: salts, buffers, preservatives, solubility enhancers, viscosity modulators, colorants, etc. Suitable aqueous vehicles include sterile distilled or purified water, isotonic solutions such as isotonic sodium chloride or boric acid solutions, phosphate buffered saline (PBS), propylene glycol and butylene glycol. Other suitable vehicular constituents include phenylmercuric nitrate, sodium sulfate, sodium sulfite, sodium phosphate and monosodium phosphate. Additional examples of other suitable vehicle ingredients include alcohols, fats and oils, polymers, surfactants, fatty acids, viscosity modifiers, emulsifiers and stabilizers, antimicrobial agents, pH adjusting agents. The viscosity of a given active agent composition may vary.

### DETAILED DESCRIPTION

The invention described herein relates to a prodrug that is converted into a dopamine agonist. The dopamine prodrug may be combined with one or more dopamine antagonists, dopamine receptor inhibitors, or vesicular monoamine transport inhibitors. A prodrug is a biologically inactive compound that can be metabolized by the body into its active form. For example, a prodrug placed on the ocular surface can undergo hydrolysis during penetration of the ocular surface, resulting in a biologically active drug. A prodrug allows for use of a lower concentration of the drug and protects against undesirable side effects. A prodrug can also help augment drug uptake into the target tissue.

To further aid in increasing dopamine levels in a target tissue, a dopamine agonist prodrug can be combined with an agent that prevents or slows the reuptake, release, or degradation of dopamine These include, but are not limited to, dopamine reuptake inhibitor and vesicular monoamine transport inhibitors.

In some embodiments, the pharmaceutical composition includes one or more of the following:

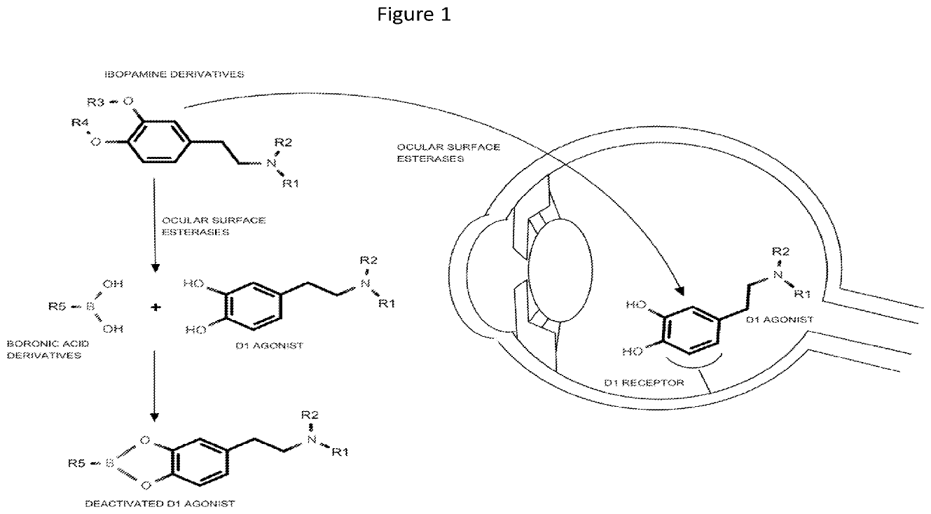

1. Dopaminergic prodrug with an agent, e.g., a binding agent, which inactivates any drug converted prior to entering the eye. In one embodiment, the binding agent comprises boronic acid (FIG. 1), which can inactive a dopamine agonist prodrug that is converted by ocular surface esterases prior to entry into the eye. The conversion then renders a drug with negative side effects to be inactive or diminished, improving the ocular or systemic side effect profile.

2. Dopaminergic prodrug with one or more dopaminergic antagonists

3. Dopaminergic prodrug with one or more dopaminergic antagonists with a binding agent which renders any drug converted prior to entering the eye inactive

4. Dopaminergic prodrug with one or more dopamine receptor inhibitors

5. Dopaminergic prodrug with one or more vesicular monoamine transport inhibitors, including reserpine

6. Etilevodopa, including etilevodopa hydrochloride, with a modified R group

7. Etilevodopa combined with an anticholinergic of choice

8. Etilevodopa combined with one or more dopamine receptor inhibitors

9. Etilevodopa combined with one or more vesicular monoamine transport inhibitors

10. Adrogolide, including adrogolide hydrochloride, with a modified R group

11. Adrogolide combined with an anticholinergic of choice

12. Adrogolide combined with one or more dopamine receptor inhibitors

13. Adrogolide combined with one or more vesicular monoamine transport inhibitors

14. Docarpamine with a modified R group

15. Docarpamine combined with an anticholinergic of choice

16. Docarpamine combined with one or more dopamine receptor inhibitors

17. Docarpamine combined with one or more vesicular monoamine transport inhibitors

18. Ibopamine with a modified R group

19. Ibopamine combined with an anticholinergic of choice

20. New chemical entity with an anticholinergic of choice. In some embodiments, the anti-cholinergic agent comprises atropine at doses including, but not limited to, 0.001% to 2%, including 0.01% to 0.05%, 0.01% to 1%, and 0.01% to 0.25%. In some embodiments, the anti-cholinergic agent comprises tropicamide at doses including, but not limited to, 0.001% to 2%, including 0.01% to 0.05%, 0.01% to 1%, and 0.1% to 0.5%.

21. Ibopamine maleate modified to increased lipophilicity and viscosity to increase ‘dwell time’ on the eye

In some embodiments, the formulation includes an ophthalmic solution comprising ibopamine and boric acid (FIG. 1). In some embodiments, ibopamine is at a concentration including, but not limited to 0.001% to 50%, including 0.01% to 0.05%, 0.01% to 1%, 1% to 5%, 5% to 10%, and including 1% to 3% including 2%. In some embodiments, boric acid is included at 0.005% to 10%.

Another ophthalmic formulation according to an embodiment of the invention includes:
ibopamine with a modified R group
boric acid

In some embodiments, the formulation includes an ophthalmic solution comprising ibopamine, boric acid, and a dopamine receptor D2 agonist.

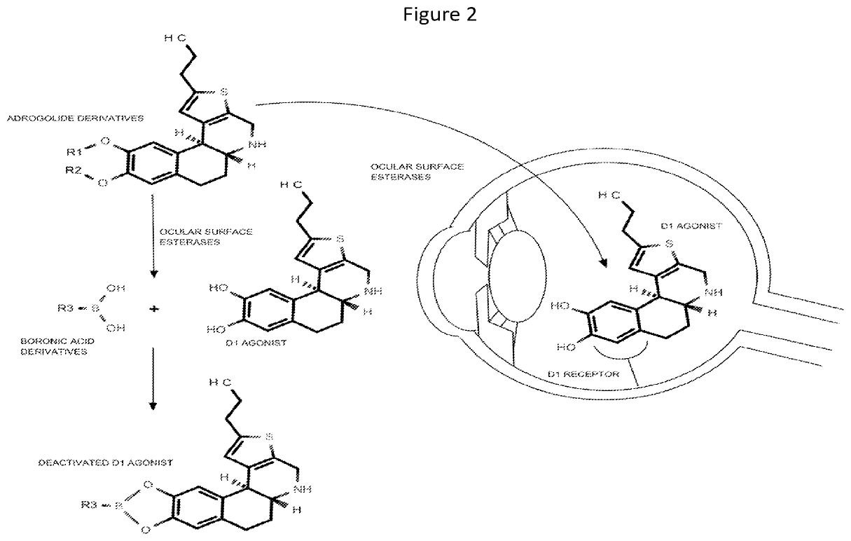

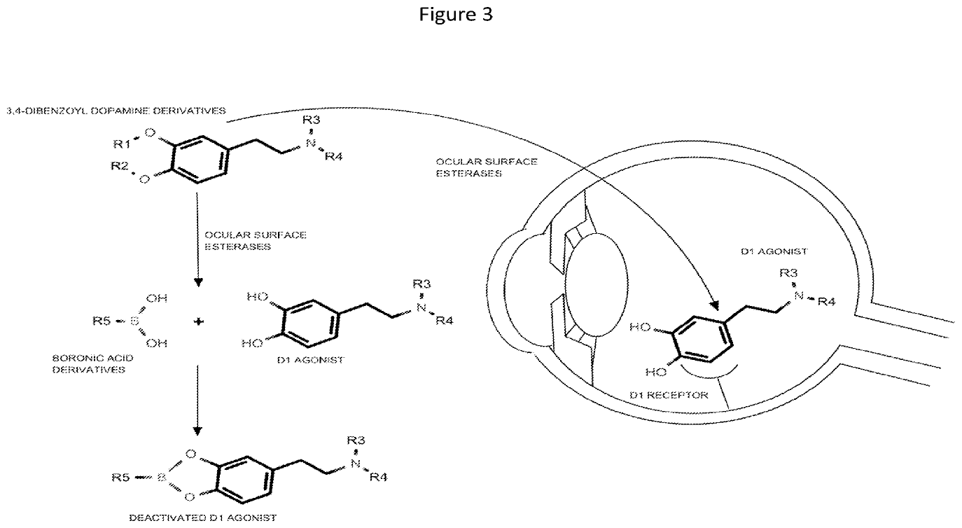

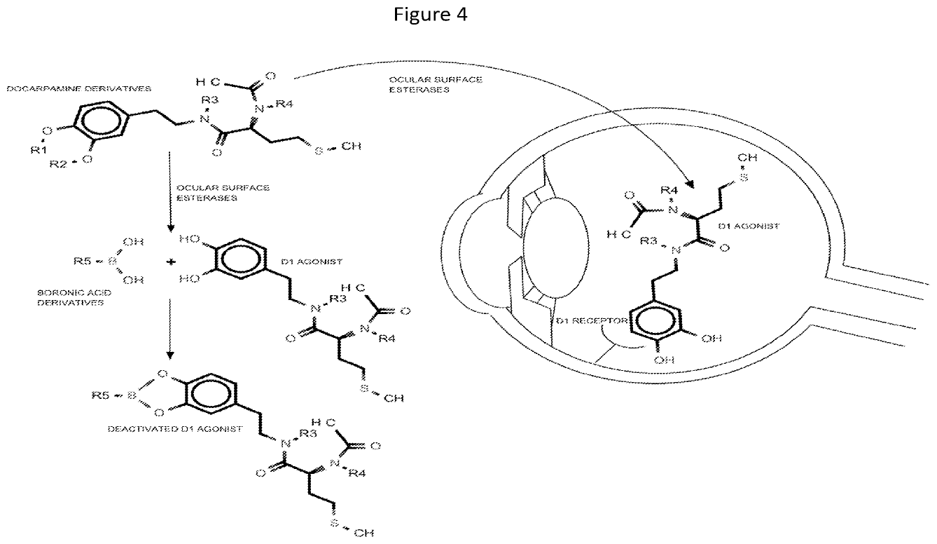

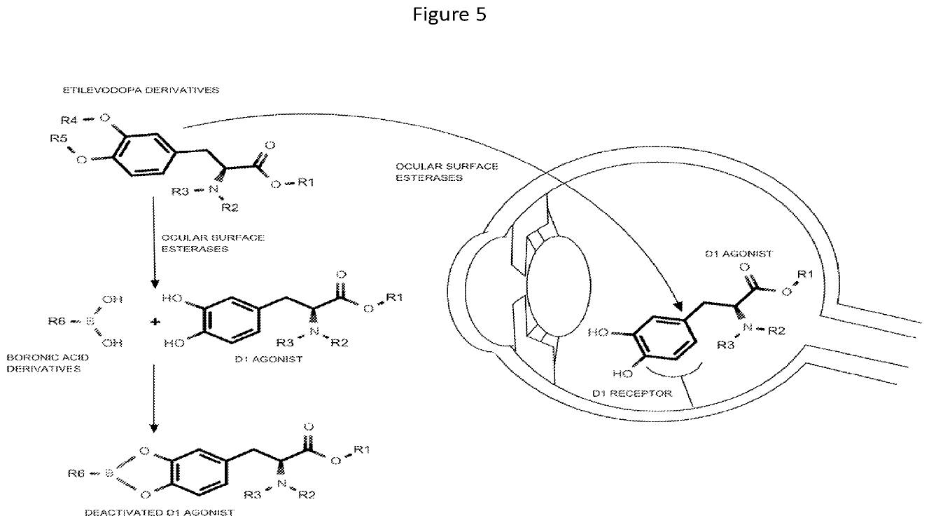

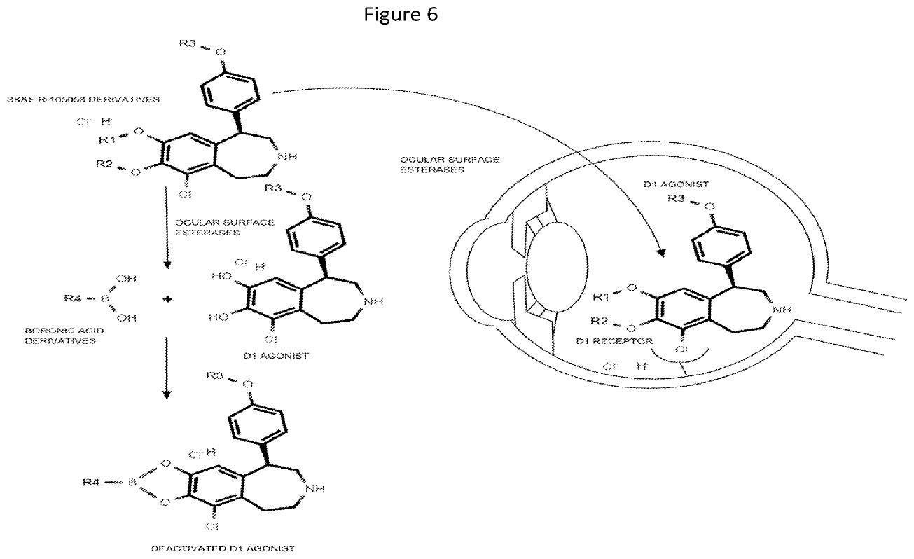

In some embodiments, the formulation includes an ophthalmic solution comprising adrogolide and boric acid (FIG. 2). In some embodiments, the formulation includes an ophthalmic solution comprising 3,4-Dibenzole dopamine derivatives and boric acid (FIG. 3). In some embodiments, the formulation includes an ophthalmic solution comprising docarpamine and boric acid (FIG. 4). In some embodiments, the formulation includes an ophthalmic solution comprising docarpamine and boric acid (FIG. 5). In some embodiments, the formulation includes an ophthalmic solution comprising SK&F R-105058 dopamine derivatives and boric acid (FIG. 6).

In some embodiments, the formulation includes an ophthalmic solution comprising etilevodopa or etilevodopa hydrochloride and boric acid. In some embodiments, etilevodopa is at a concentration including, but not limited to 0.0001% to 50%, including 0.01% to 0.05%, 0.01% to 1%, 1% to 5%, 5% to 10%, and including 1% to 3% including 2%. In some embodiments, boric acid is included at 0.005% to 10%.

Another ophthalmic formulation according to embodiments of the invention includes:
etilevodopa with a modified R group
boric acid

In some embodiments, the formulation includes an ophthalmic solution comprising etilevodopa, boric acid, and a dopamine receptor D2 agonist.

In some embodiments, the formulation includes an ophthalmic solution comprising adrogolide and boric acid. In some embodiments, adrogolide or adrogolide hydrochloride is at a concentration including, but not limited to 0.0001% to 50%, including 0.01% to 0.05%, 0.01% to 1%, 1% to 5%, 5% to 10%, and including 1% to 3% including 2%. In some embodiments, boric acid is included at 0.005% to 10%.

Another ophthalmic formulation according to embodiments of the invention includes:
adrogolide or adrogolide hydrochloride with a modified R group
boric acid

In some embodiments, the formulation includes an ophthalmic solution comprising adrogolide, boric acid, and a dopamine receptor D2 agonist.

In some embodiments, the formulation includes an ophthalmic solution comprising Docarpamine and boric acid. In some embodiments, docarpamine hydrochloride is at a concentration including, but not limited to 0.0001% to 50%, including 0.01% to 0.05%, 0.01% to 1%, 1% to 5%, 5% to 10%, and including 1% to 3% including 2%. In some embodiments, boric acid is included at 0.005% to 10%.

Another ophthalmic formulation according to embodiments of the invention includes:
docarpamine with a modified R group
boric acid

In some embodiments, any of the dopamine receptor agonist prodrugs listed herein, either alone, or in combination, may be present at a concentration including, but not limited to 0.0001% to 50%, including 0.01% to 0.05%, 0.01% to 1%, 1% to 5%, 5% to 10%, and including 1% to 3% including 2%.

In some embodiments, these formulations include boric acid at 0.005% to 10%.

In some embodiments, the formulation includes an ophthalmic solution comprising docarpamine, boric acid, and a dopamine receptor D2 agonist.

In some embodiments, the formulation includes an ophthalmic solution comprising one or more of the dopamine prodrugs described elsewhere in this specification, further comprising boric acid, and a vesicular monoamine transport inhibitor.

In some embodiments, the formulation includes an ophthalmic solution comprising one or more of the dopamine prodrugs described elsewhere in this specification, further comprising boric acid, and a dopamine reuptake inhibitor.

In some embodiments, the formulation includes a dopamine receptor inhibitor or prodrug thereof as a sole active agent. In some embodiments, the formulation consists of a vesicular monoamine transport inhibitor or prodrug thereof as a sole active agent.

As described in Barot et al. in 2012, the common functional groups in ophthalmic prodrug design are carboxylic, hydroxyl, amine, and carbonyl groups. Modification of these functional groups (esters, carbamates, phosphates, and oximes) results in ophthalmic prodrugs. Prodrug structures of the most common moieties as described by Barot et al. are as follows:

TABLE 1
Common functional groups in ophthalmic prodrug design (Barot et al. 2012)

| | | | | Hydrolytic | |
|---|---|---|---|---|---|
| Prodrug | Functional Groups | Prodrug Form | Drugs | Enzyme | Comments |
| Ester | —OH —COOH | 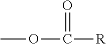 | Antimetabolites Prostaglandins | Esterases | Enhanced corneal penetration & IOP reduction, chemical |
| | | | | | instability in aqueous |
| | | | | | solution |
| Phosphate Ester | —OH | 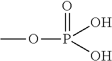 | Vidarabine Cannabinoids | Phosphatases | Enhanced aqueous solubility, excellent chemical stability and high suscept- |
| | | | | | ibility to enzymatic |
| | | | | | reconversion |
| Carbamate Ester | —NH2 | 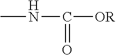 | Timolol | Esterases | Enhanced corneal penetration, high enzymatic stability |
| Oxime | 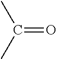 | 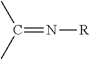 | Beta-adrenergic blockers | Oxime hydrolase, ketone reductase | Prolonged IOP reduction and improved therapeutic index, site-specific |
| | | | | | enzyme activity |
| Oxazolidines | 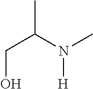 | 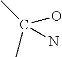 | Phenylephrine | — | Enhanced corneal penetration and improved therapeutic index, poor aqueous stability |
| N-sulfonyl imidates | —SO2NH2 | 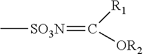 | Carbonic anhydrase inhibitors | — | Enhanced aqueous solubility, slow hydrolytic rate and poor aqueous |
| | | | | | stability |

The COOH and OH functional groups of ester prodrugs exist in an ionized form under physiologic conditions which does not favor passage through lipid membranes yielding poor drug bioavailability.

However, when hydrolyzed by esterases such as acetylcholine esterase, pseudocholine esterase, butyry-choline esterase and carboxyl esterase, these drugs may penetrate the cornea in their active form.

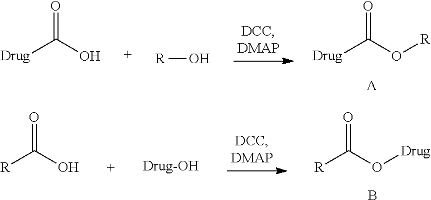

Examples of R Groups include H, COCH3 (monoacetate), COCH2CH3 (monopropionate), CO(CH2)2CH3 (monobutyrate), COC(CH3)3, CH3, CH(CH3)2, C(CH3)3, C6H5, C2H50.

Examples of ester prodrugs with COOH and OH functional groups currently used in ophthalmology include the following:

TABLE 2
OH Functionalities.

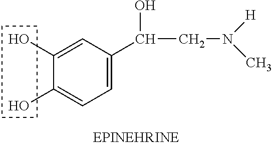

Dipivefrin

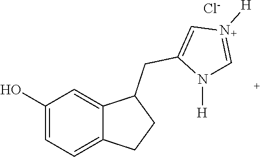

Fadolmidine

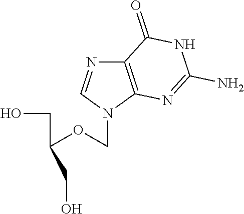

Ganciclovir

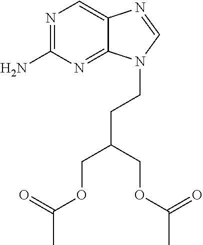 [SMILES: CC(=O)OCC(CCn1cnc2cnc(N)nc21)COC(C)=O]

Famciclovir

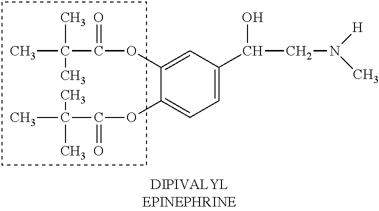 [SMILES: CNCC(O)c1ccc(OC(=O)C(C)(C)C)c(OC(=O)C(C)(C)C)c1]

Epinephrine

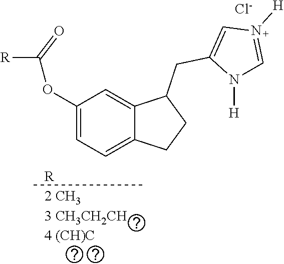

Fadolmidine

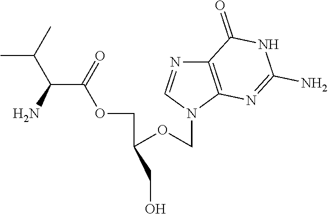 [SMILES: CC(C)[C@H](N)C(=O)OC[C@H](CO)OCn1cnc2c(=O)[nH]c(N)nc21]

Valganciclovir

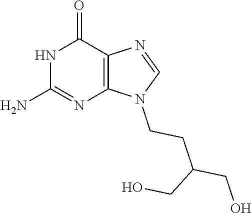 [SMILES: Nc1nc2c(ncn2CCC(CO)CO)c(=O)[nH]1]

Penciclovir

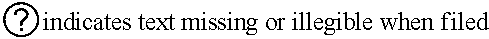

TABLE 3
COOH Functionalities.

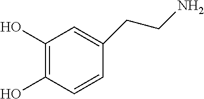 [SMILES: NCCc1ccc(O)c(O)c1]

Dopamine

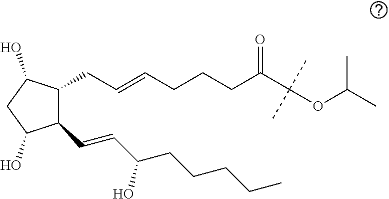

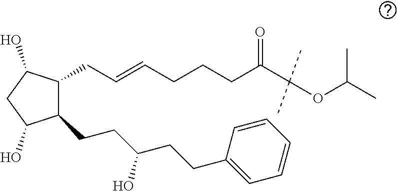

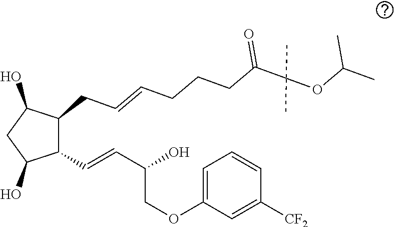

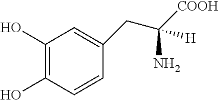 [SMILES: N[C@@H](Cc1ccc(O)c(O)c1)C(=O)O]

Levodopa

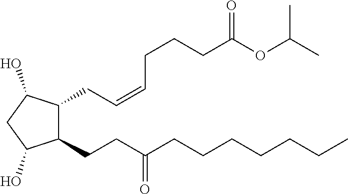 [SMILES: CCCCCCCC(=O)CC[C@@H]1[C@@H](C/C=C\CCCC(=O)OC(C)C)[C@@H](O)C[C@H]1O]

Unoprostone isopropyl

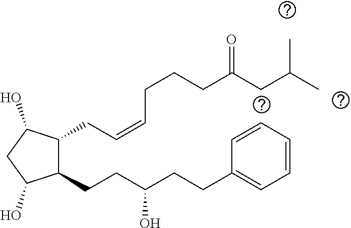 [SMILES: CC(C)CC(=O)CCC/C=C\C[C@@H]1[C@@H](CC[C@@H](O)CCc2ccccc2)[C@H](O)C[C@@H]1O]

Latanoprost

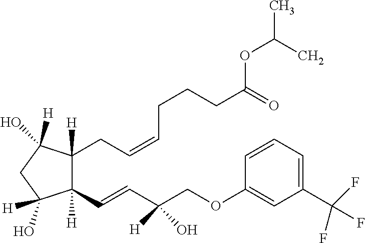 [SMILES: CC(C)OC(=O)CCC/C=C\C[C@@H]1[C@@H](/C=C/[C@@H](O)COc2cccc(C(F)(F)F)c2)[C@H](O)C[C@@H]1O]

Travoprost

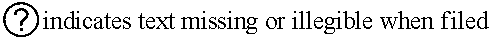

Additionally, new topical anti-VEGF agents are being evaluated that use cell-penetrating peptides to enter the eye. These short peptides allow for VEGFs to gain access to the interior of cells. Some topical anti-VEGF agents contain both hydrophilic and hydrophobic components that allow the drug to penetrate the ocular surface. An example of this mechanism exists in squalamine, a natural aminosterol with a flexible polyamino-hydrophilic spermidine group linked to the hydrophobic unit at the C-3 position that allows it to penetrate the ocular surface. These mechanisms may be used in the invention described herein to allow for better penetration of the surface of the eye.

### Dopamine Agents

Dopamine interacts with two major types of receptors, the D1 and D2 receptors. In some embodiments, a dopamine prodrug includes, but is not limited to a new/modified chemical entity that after interaction with corneal esterases binds D1 and/or D2, ibopamine (including ibopamine hydrochloride or ibopamine maleate), or etilevodopa. The dopaminergic agent within the invention may also include any of the following: (+)-PD 128,907 hydrochloride, (±)-6-Chloro-PB hydrobromide, (±)-7-Hydroxy-2-(di-n-propylamino)tetralin hydrobromide, (±)-PD 128,907 hydrochloride solid, (±)-SKF-38393 hydrochloride, (R)-(+)-SKF-38393 hydrochloride, (R)-SKF-82957 hydrobromide solid, 2-Bromo-α-ergocryptine methanesulfonate salt solid, 6,7-ADTN hydrobromide, 68930 Hydrochloride, A-412997 dihydrochloride, A-77636 hydrochloride hydrate, ABT-724 trihydrochloride, Amantadine, Apomorphine, Aripiprazole, Bifeprunox mesylate, BP897, Brexpiprazole, Bromocriptine, Cabergoline, Cabergoline , Cariprazine, Chloro-APB hydrobromide solid, cis-8-Hydroxy-3-(n-propyl)-1,2,3a,4,5,9b-hexahydro-1H-benz[e]indole hydrobromide , CJB 090 dihydrochloride hydrate, CP-226269, Dexpramipexole, Dexpramipexole dihydrochloride, Dihydrexidine, Dihydro-alpha, Dihydroergocornine,

Dihydroergocryptine, Dihydroergotamine, Dopamine, Dopamine hydrochloride, Dopexamine, Ergoloid mesylate, Ergotamine, Fenoldopam, Fenoldopam mesylate, Fenoldopam monohydrobromide, FFN102 , FFN511 trifluoroacetate salt hydrate, Keramine, Levodopa, Lisuride, Lumateperone, Metergoline, Minaprine, MLS1547, N-Allyl-(±)-SKF-38393 hydrobromide solid, Olanzapine, OS-3-106, OSU6162 hydrochloride, PD 168,077 maleate salt powder, Pergolide, Phenylpropanolamine, Piribedil, Piribedil maleate salt, Pramipexole, Pramipexole dihydrochloride, Quinagolide, Quinpirole, Quinpirole hydrochloride, R-(−)-Apomorphine hydrochloride hemihydrate calcined, R(+)-3-(3-Hydroxyphenyl)-N-propylpiperidine hydrochloride solid, R(+)-6-Bromo-APB hydrobromide solid, R(+)-SKF-81297 hydrobromide, Ropinirole, Ropinirole hydrochloride powder, Rotigotine, Rotigotine hydrochloride, S-(−)-SKF-38393 hydrochloride solid, Salsolinol hydrobromide, SB269652 , SKF83822 hydrobromide, SKF-83959 hydrobromide, SKF-89145 hydrobromide, Sumanirole maleate, or Tyramine hydrochloride. Dopaminergic prodrugs include, but are not limited to: dopamine prodrugs, levodopa prodrugs, and dopamine receptor agonist prodrugs.

Dopamine prodrugs include but are not limited to: ibopamine and 3,4-dibenzoyl dopamine, in addition to the lipophilic 3,4-O-diesters, pyridinium/dihydropyridine redox carriers, 1-methyl-3-[N-(-(β-3,4-dihydroxyphenyl)ethyl)carbamoil]pyridinium salt, glycoconjugates, glycosyl derivatives of dopamine mentioned by Di Stefano et al. (Molecules. 2008).

Levodopa prodrugs include, but are not limited to: etilevodopa in addition to the series of di- and tripeptids containing levodopa, the tripeptide mimetic dopaminergic prodrug in which D-p-hydroxyphenylglycine-L-proline was attached to levodopa, classes of transient derivatives of levodopa, glycosyl derivatives of levodopa, AADC1-3-(3-hydroxy-4-pivaloyloxybenzyl)-2,5-diketomorpholine, dimeric derivatives of levodopa diacetyl esters, a codrug in which levodopa and entacapone are linked via a biodegradable carbamate spacer to form a single chemical entity, dual acting codrugs in which LD and benserazide are covalently coupled together by different oxalyl and carbonyl spacers, novel molecular combinations in which levodopa and dopamine are linked to antioxidant and iron-chelating agents such as (R)-α-lipoic acid (LA) and glutathione mentioned by Di Stefano et al. (Molecules. 2008).

Dopamine receptor agonist prodrugs include but are not limited to: adrogolide, adrogolide hydrochloride, SK&F R-105058, docarpamine, in addition to series of the O, O′-diesters of apomorphine, 2-amino-6,7-dihydroxytetrahydronaphthalene (ADTN), a dibenzoyl-ester derivative of 6,7-ADTN, a series of ether derivatives of the purported DA agonist 3,4-dihydroxyphenylimino-2-imidazolidine (DPI) and the diphenylmethane ether analogue, derivatives bearing substituents on the phenolic function of (−)-3-(3-hydroxyphenyl)-N-propylpiperidine [(−)-3-PPP], N-0437 [2-(N-propyl-N-2-thienylethylamino)-5-hydroxy-tetralin, [(−)-(5aR,11bS)-4,5,-5a,6,7,11b-hexahydro-2-propyl-3-thia-5-azacyclopent-fena[c]phenanthrene-9,10-diol], A-869293 and its diacetyl prodrug derivative (ABT-431), S-(−)-6-(N,N-di-n-propylamino)-3,4,5,6,7,8-hexahydro-2H-naphthalen-1-one [(−)-(S-PD148903)], 5-5,6-di0H-DPAT, the series of oxime derivatives of the dopaminergic prodrug S-PD148903, and the benzo[g]quinoline-derived enones mentioned by Di Stefano et al. (Molecules. 2008).

TABLE 4
Dopamine receptor D1 agonists

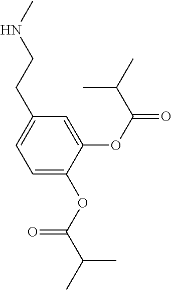 [SMILES: CNCCc1ccc(OC(=O)C(C)C)c(OC(=O)C(C)C)c1]

Ibopamine

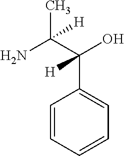

Phenylpropanolamine

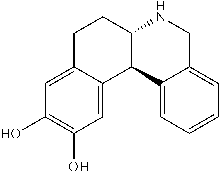 [SMILES: Oc1cc2c(cc1O)[C@@H]1c3ccccc3CN[C@H]1CC2]

Dihydrexidine

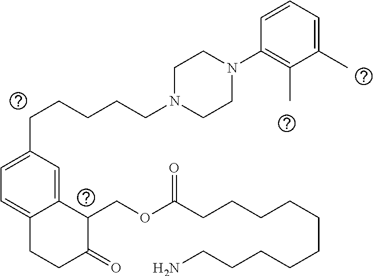 [SMILES: Cc1cccc(N2CCN(CCCCCc3ccc4c(c3)C(COC(=O)CCCCCCCCCCN)C(=O)CC4)CC2)c1C]

Aripiprazole Lauroxil

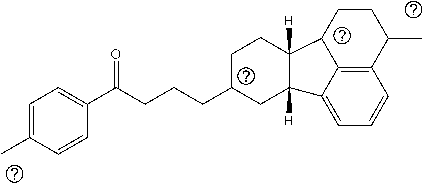

Lumateperone

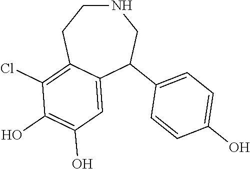 [SMILES: Oc1ccc(C2CNCCc3c2cc(O)c(O)c3Cl)cc1]

Fenoldopam

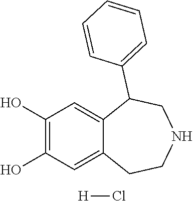

SKF-38393

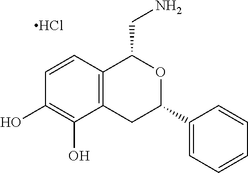 [SMILES: NC[C@@H]1O[C@H](c2ccccc2)Cc2c1ccc(O)c2O ; Cl]

68930 Hydrochloride

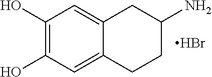 [SMILES: NC1CCc2cc(O)c(O)cc2C1 ; Br]

6,7-ADTN hydrobromide

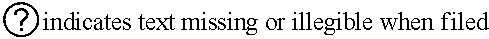

TABLE 5
Dopamine receptor D2 agonists

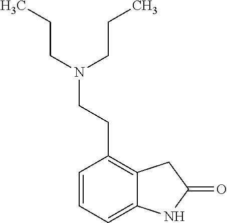 [SMILES: CCCN(CCC)CCc1cccc2c1CC(=O)N2]

Ropinirole

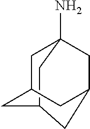 [SMILES: NC12CC3CC(CC(C3)C1)C2]

Amantadine

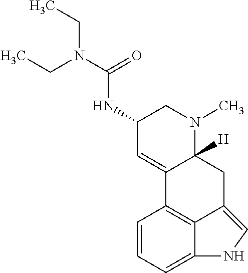 [SMILES: CCN(CC)C(=O)N[C@H]1C=C2c3cccc4[nH]cc(c34)C[C@H]2N(C)C1]

Lisuride

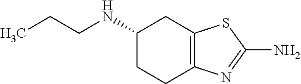

Pramipexole

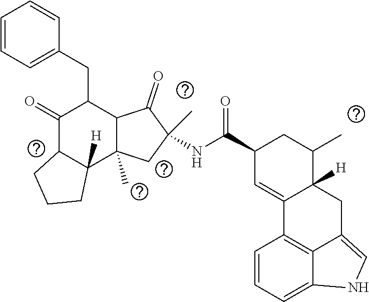

Ergotamine

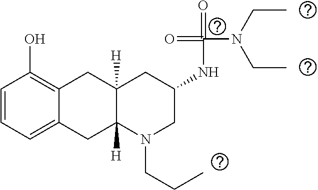

Quinagolide

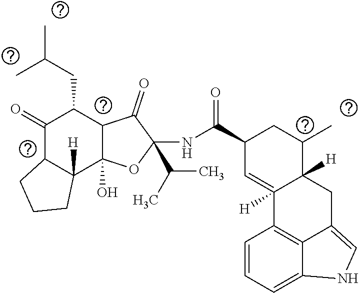

Dihydro-alpha

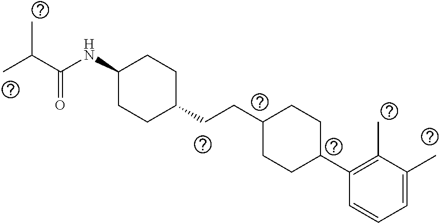 [SMILES: Cc1cccc(C2CCC(CC[C@H]3CC[C@H](NC(=O)C(C)C)CC3)CC2)c1C]

Cariprazine

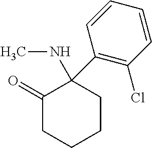 [SMILES: CNC1(c2ccccc2Cl)CCCCC1=O]

Keramine

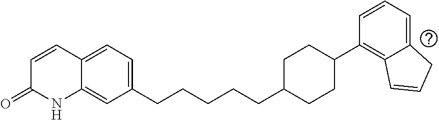 [SMILES: O=c1ccc2ccc(CCCCCC3CCC(c4cccc5c4C=CC5)CC3)cc2[nH]1]

Brexpiprazole

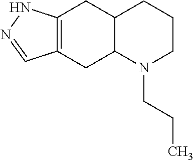 [SMILES: CCCN1CCCC2Cc3[nH]ncc3CC21]

Quinpirole

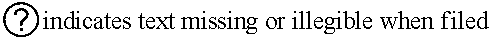

TABLE 6
Mixed dopamine receptor D1 and D2 agonists

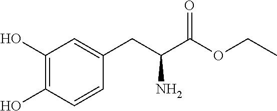 [SMILES: CCOC(=O)[C@@H](N)Cc1ccc(O)c(O)c1]

Etilevodopa

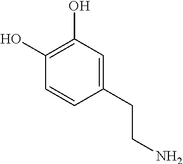 [SMILES: NCCc1ccc(O)c(O)c1]

Dopamine

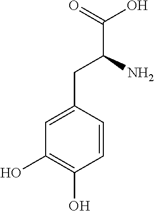 [SMILES: N[C@@H](Cc1ccc(O)c(O)c1)C(=O)O]

Levodopa

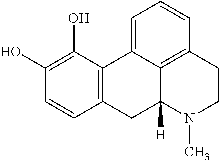 [SMILES: CN1CCc2cccc3c2[C@H]1Cc1ccc(O)c(O)c1-3]

Apomorphine

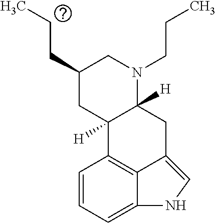 [SMILES: CCC[C@@H]1C[C@@H]2c3cccc4[nH]cc(c34)C[C@H]2N(CCC)C1]

Pergolide

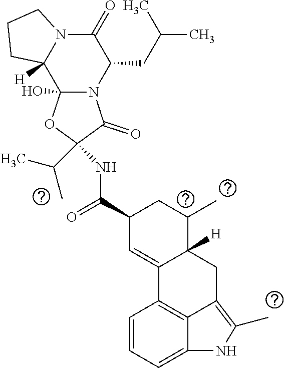 [SMILES: Cc1[nH]c2cccc3c2c1C[C@@H]1C3=C[C@@H](C(=O)N[C@]2(C(C)C)O[C@@]3(O)[C@@H]4CCCN4C(=O)[C@H](CC(C)C)N3C2=O)CC1C]

Bromocriptine

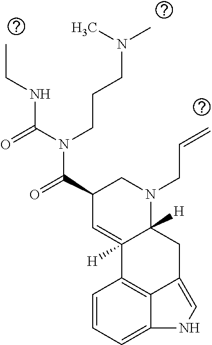

Cabergoline

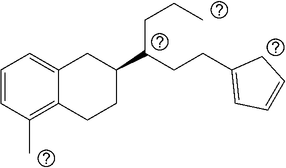 [SMILES: CCCC(CCC1=CC=CC1)[C@H]1CCc2c(C)cccc2C1]

Rotigotine

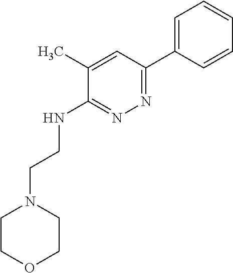 [SMILES: Cc1cc(-c2ccccc2)nnc1NCCN1CCOCC1]

Minarprine

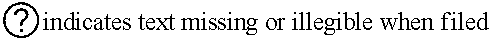

In some embodiments, a dopamine prodrug is combined with an antidopaminergic agent, including, but not limited to acepromazine, aceprometazine, acetophenazine, alizapride, amisulpride, amoxapine, aripiprazole, as-8112, asenapine, azaperone, benperidol, blonanserin, bromopride, buspirone, carphenazine, chlorpromazine, chlorprothixene, clebopride, clopenthixol, clozapine, domperidone, doxepin, droperidol, ecopipam, eticlopride, fallypride, flupentixol, fluphenazine, fluspirilene, haloperidol, iloperidone, jnj-37822681, lisuride, loxapine, lumateperone, lurasidone, melperone, mesoridazine, methotrimeprazine, methylergometrine, metoclopramide, mianserin, molindone, nortriptyline, olanzapine, paliperidone, penfluridol, perazine, periciazine, perospirone, perphenazine, pimozide, pipamperone, pipotiazine, prochlorperazine, promazine, promethazine, propiomazine, quetiapine, raclopride, remoxipride, risperidone, sertindole, sulpiride, sultopride, tetrahydropalmatine, thiethylperazine, thioproperazine, thioridazine, thiothixene, tiapride, trifluoperazine, trifluperidol, triflupromazine, ykp-1358, yohimbine, ziprasidone, zotepine, or zuclopenthixol.

### Miotic Agents

In some embodiments, a dopamine prodrug is combined with a miotic agent, including, but not limited to aceclidine, acetylcholine, carbamoylcholine, echothiophate, methacholine, moxisylyte, physostigmine, or pilocarpine.

### Anticholinergic Agents

In some embodiments, a dopamine prodrug is combined with an anticholinergic agent, including, but not limited to aclidinium, agmatine, alcuronium, amantadine, amitriptyline, amobarbital, amoxapine, anisotropine methylbromide, aprobarbital, aripiprazole, atracurium, atracurium besylate, atropine, barbital, barbituric acid derivative, batefenterol, benactyzine, benzatropine, benzilone, benzquinamide, bevonium, biperiden, bornaprine, brompheniramine, buclizine, butabarbital, butalbital, butobarbital, butylscopolamine, camylofin, chloroprocaine, chlorphenoxamine, chlorpromazine, chlorprothixene, cisatracurium, clidinium, clozapine, cocaine, cyclopentolate, cycrimine, cyproheptadine, darifenacin, desipramine, desloratadine, dexetimide, dextromethorphan, dicyclomine, difemerine, dihexyverine, dimetindene, diphemanil, diphenhydramine, diphenidol, disopyramide, dosulepin, doxacurium, doxepin, doxylamine, emepronium, emetonium iodide, escitalopram, etanautine, etybenzatropine, fenoterol, fenpiverinium, fesoterodine, flavoxate, fluoxetine, flupentixol, gallamine, gallamine triethiodide, gantacurium, glycopyrronium, heptabarbital, hexafluronium, hexamethonium, hexobarbital, hexocyclium, homatropine, homatropine methylbromide, hyoscyamine, imidafenacin, imipramine, ipratropium, isoflurane, isopropamide, lamotrigine, levacetylmethadol, maprotiline, mazaticol, mebeverine, mecamylamine, mepenzolate, methadone, methantheline, metharbital, methotrimeprazine, methscopolamine, methscopolamine bromide, methylphenobarbital, metixene, metocurine, metocurine iodide, mivacurium, nicardipine, nortriptyline, olanzapine, orphenadrine, otilonium, oxitropium, oxybutynin, oxyphencyclimine, oxyphenonium, pancuronium, paroxetine, penthienate, pentobarbital, pentolinium, phenglutarimide, phenobarbital, pipecuronium, pipenzolate, piperidolate, pirenzepine, pizotifen, poldine, prifinium, primidone, procaine, procyclidine, profenamine, promazine, promethazine, propantheline, propiomazine, propiverine, quetiapine, quinidine, rapacuronium, revefenacin, rociverine, rocuronium, scopolamine, secobarbital, solifenacin, talbutal, terfenadine, thiopental, thonzylamine, tiemonium iodide, timepidium, tiotropium, tolterodine, tramadol, tridihexethyl, triflupromazine, trihexyphenidyl, trimebutine, trimethaphan, tropatepine, tropicamide, trospium, tubocurarine, umeclidinium, vecuronium, or ziprasidone.

Anticholinergics have been studied in the ATOM, ATOM2 and LAMP studies and may act to thicken the sclera. This combined with a dopaminergic agent which may act upstream of the effects of anticholinergics may result in compounded benefits in inhibiting the progression of myopia.

### Vesicular Monoamine Transport (VMAT) Inhibitors

In some embodiments, a dopamine prodrug is combined with a VMAT inhibitor, including, but not limited to reserpine, bietaserpine, ketanserin, tetrabenazine, deutetrabenazine, phenylethylamine, amphetamine, N-methyl-4phenylpyridinium, fenfluramine, non-dyrolysable GTP-analogue guanylyllimidodiphosphate. In some embodiments, the VMAT inhibitor is a prodrug, including, but not limited to, Valbenazine. In some embodiments, the VMAT inhibitor comprises the sole active agent. In some embodiments, the VMAT inhibitor prodrug is combined with a dopamine prodrug. In some embodiments, the VMAT inhibitor prodrug is combined with a dopamine prodrug and a dopamine reuptake inhibitor.

### Dopamine Reuptake Inhibitors (DRI)

In some embodiments, a dopamine prodrug is combined with a DRI, including, but not limited to 4-Hydroxy-1-methyl-4-(4-methylphenyl)-3-piperidyl 4-methylphenyl ketone, altropane, amfonelic acid, amantadine, adrafinil, amineptine, benztropine, bupropion, cocaine, fluorenol, medifoxamine, BTCP, 3C-PEP, DBL-583, difuoropine, GBR-12783, GBR-12935, GBR-13069, GBR-13098, GYKI-52895, iometopane, metaphit, methylphenidate, ethylphenidate, modafinil, armodafinil, rimcazole, RTI-229, vanoxerine, venflafaxine. In some embodiments, a DRI is administered as the sole agent. In some embodiments, a dopamine reuptake inhibitor prodrug is administered as the sole agent. In some embodiments, a DRI prodrug is combined with a dopamine prodrug to increase the concentration of dopamine in a biological tissue. In some embodiments, the biological tissue comprises the eye.

### IOP Lowering Agents

In some embodiments, a dopamine prodrug is combined with an intra-ocular pressure (TOP) lowering agent. Non-limiting examples of TOP agents that may be employed in some embodiments are as follows.

In some embodiments, a dopamine prodrug is combined with an alpha agonist, including, but not limited to 4-bromo-2,5-dimethoxyphenethylamine, 4-methoxyamphetamine, adrafinil, amitraz, apraclonidine, benzphetamine, bethanidine, brimonidine, bromocriptine, cirazoline, clonidine, detomidine, dexmedetomidine, dipivefrin, dl-methylephedrine, dobutamine, droxidopa, ephedra sinica root, ephedrine, epinephrine, ergometrine, etilefrine, etomidate, guanabenz, guanfacine, isometheptene, lofexidine, medetomidine, mephentermine, metamfetamine, metaraminol, methoxamine, methyldopa, midodrine, moxonidine, naphazoline, norepinephrine, norfenefrine, octopamine, oxymetazoline, pergolide, phendimetrazine, phenylephrine, phenylpropanolamine, pseudoephedrine, racepinephrine, rilmenidine, romifidine, synephrine, tetryzoline, tizanidine, xylazine, or xylometazoline

In some embodiments, a dopamine prodrug is combined with a beta blocker, including, but not limited to acebutolol, alprenolol, anisodamine, arotinolol, atenolol, befunolol, betaxolol, bevantolol, bisoprolol, bopindolol, bucindolol, bufuralol, bupranolol, carteolol, carvedilol, celiprolol, cloranolol, dexpropranolol, epanolol, esatenolol, esmolol, indenolol, labetalol, landiolol, levobetaxolol, levobunolol, mepindolol, metipranolol, metoprolol, nadolol, nebivolol, oxprenolol, penbutolol, pindolol, practolol, propafenone, propranolol, sotalol, talinolol, tertatolol, or timolol.

In some embodiments, a dopamine prodrug is combined with a carbonic anhydrase inhibitor, including, but not limited to acetazolamide, brinzolamide, diclofenamide, dorzolamide, ethoxzolamide, indisulam, methazolamide, or zonisamide.

In some embodiments, a dopamine prodrug is combined with a cholinergic agent, including, but not limited to 1,10-phenanthroline, acetylcholine, acotiamide, alcuronium, ambenonium, anisotropine methylbromide, arecoline, atracurium, atracurium besylate, atropine, benactyzine, benzatropine, bethanechol, biperiden, bornaprine, botulinum toxin type a, botulinum toxin type b, butylscopolamine, carbamoylcholine, cevimeline, chlorphenoxamine, coumaphos, cyclopentolate, darifenacin, demecarium, desloratadine, dexetimide, dichlorvos, dicyclomine, distigmine, donepezil, echothiophate, edrophonium, emepronium, epibatidine, fenthion, fesoterodine, galantamine, gallamine, gallamine triethiodide, glycopyrronium, gts-21, hexafluronium, hexamethonium, huperzine a, hyoscyamine, imidacloprid, ipratropium, isoflurophate, lobeline, malathion, mecamylamine, methacholine, methanesulfonyl fluoride, methantheline, metrifonate, mivacurium, neostigmine, ngx267, nicotine, obidoxime, orphenadrine, otilonium, oxyphenonium, pancuronium, paraoxon, pentolinium, phenglutarimide, phenserine, physostigmine, pilocarpine, pipecuronium, pirenzepine, posiphen, pralidoxime, procyclidine, profenamine, propantheline, propiverine, pyridostigmine, quinidine, rivastigmine, scopolamine, solifenacin, tacrine, tiotropium, tolterodine, trihexyphenidyl, trimethaphan, tropicamide, trospium, tubocurarine, varenicline, vecuronium, or xanomeline

In some embodiments, a dopamine prodrug is combined with a prostaglandin analogue, including, but not limited to alprostadil, beraprost, bimatoprost, carboprost tromethamine, cloprostenol, dinoprost, dinoprost tromethamine, dinoprostone, enprostil, epoprostenol, fenprostalene, fluprostenol, gemeprost, iloprost, latanoprost, latanoprostene bunod, limaprost, luprostiol, misoprostol, prostaglandin b2, prostaglandin d2, prostaglandin g2, prostalene, sulprostone, tafluprost, travoprost, treprostinil, or unoprostone.

In some embodiments, a dopamine prodrug is combined with a rho-kinase inhibitor, including, but not limited to netarsudil.

### Anti-inflammatory Agents

In some embodiments, a dopamine prodrug is combined with an anti-inflammatory agent, such as a non-steroidal anti-inflammatory agent, including, but not limited to aceclofenac, acemetacin, acetylsalicylic acid, alclofenac, alminoprofen, aminophenazone, antipyrine, antrafenine, azapropazone, balsalazide, bendazac, benorilate, benoxaprofen, benzydamine, betulinic acid, bromfenac, bufexamac, bumadizone, carbaspirin calcium, carprofen, celecoxib, choline magnesium trisalicylate, cimicoxib, clonixin, dexibuprofen, dexketoprofen, diclofenac, difenpiramide, diflunisal, droxicam, ebselen, epirizole, ethenzamide, etodolac, etofenamate, etoricoxib, felbinac, fenbufen, fenoprofen, fentiazac, feprazone, firocoxib, floctafenine, flunixin, flunoxaprofen, flurbiprofen, flurbiprofen axetil, guacetisal, ibuprofen, ibuproxam, icosapent, imidazole salicylate, indobufen, indomethacin, indoprofen, isoxicam, kebuzone, ketoprofen, ketorolac, licofelone, lonazolac, lornoxicam, loxoprofen, lumiracoxib, magnesium salicylate, meclofenamic acid, mefenamic acid, meloxicam, mesalazine, metamizole, mofebutazone, morniflumate, nabumetone, naproxen, nepafenac, nifenazone, niflumic acid, nimesulide, nitroaspirin, ns-398, olsalazine, oxaprozin, oxyphenbutazone, parecoxib, phenylbutazone, piroxicam, pirprofen, polmacoxib, pranoprofen, proglumetacin, propacetamol, propyphenazone, proquazone, robenacoxib, rofecoxib, salicylamide, salicylic acid, salsalate, sc-236, sulfasalazine, sulindac, suprofen, suxibuzone, talniflumate, taxifolin, tenidap, tenoxicam, tepoxalin, tiaprofenic acid, tinoridine, tolfenamic acid, tolmetin, trolamine salicylate, ursolic acid, valdecoxib, zaltoprofen, or zomepirac.

### Dry Eye Agents

In some embodiments, a dopamine prodrug is combined with a drug targeting dry eye syndrome, including, but not limited to azithromycin, boscalid, botulinum toxin type A, brivudine, carboxymethylcellulose, chondroitin sulfate, cyclosporine, dexamethasone, dipyridamole, doxycycline, ecabet, fluorometholone, glycerin, hyaluronic acid, hypromellose, levofloxacin, lifitegrast, loteprednol etabonate, mineral oil, olive oil, omega-3 fatty acids, ozagrel, petrolatum, polyethylene glycol 400, povidone, prednisolone, prednisolone acetate, propylene glycol, rebamipide, tacrolimus, tavilermide, thymosin beta-4, tofacitinib, vitamin A, vitamin E, or xanthan gum.

### Anti-Allergic Agents

In some embodiments, a dopamine prodrug is combined with a drug targeting allergies, including, but not limited to alcaftadine, azelastine, bepotastine, carbinoxamine, cortisone acetate, cyproheptadine, dexamethasone, dexchlorpheniramine maleate, diclofenac, emedastine, epinastine, hydrocortisone, ketorolac, ketotifen, loteprednol, methylprednisolone, nedocromil, olopatadine, pheniramine, prednisolone, prednisone, promethazine, or triprolidine.

In some embodiments, a dopamine prodrug is combined with an antihistamine drug, including, but not limited to abt-288, aceprometazine, acrivastine, alcaftadine, alimemazine, amitriptyline, amoxapine, antazoline, aripiprazole, aripiprazole lauroxil, asenapine, astemizole, azatadine, azelastine, bamipine, benzatropine, benzquinamide, bepotastine, betahistine, bilastine, bromodiphenhydramine, brompheniramine, buclizine, butriptyline, carbinoxamine, cariprazine, cetirizine, chlorcyclizine, chloropyramine, chlorpheniramine, chlorphenoxamine, chlorpromazine, chlorprothixene, cimetidine, cinnarizine, clemastine, clofedanol, clozapine, cyclizine, cyproheptadine, deptropine, desipramine, desloratadine, dexbrompheniramine, dexchlorpheniramine, dexchlorpheniramine maleate, dimenhydrinate, dimetindene, dimetotiazine, diphenhydramine, diphenylpyraline, dosulepin, doxepin, doxylamine, ebastine, emedastine, epinastine, esmirtazapine, famotidine, fexofenadine, flunarizine, gsk-1004723, gsk-239512, hydroxyzine, iloperidone, imipramine, isothipendyl, ketotifen, lafutidine, lavoltidine, levocabastine, levocetirizine, loratadine, maprotiline, meclizine, mepyramine, mequitazine, methantheline, methapyrilene, methdilazine, methotrimeprazine, metiamide, mianserin, mirtazapine, mizolastine, niperotidine, nizatidine, nortriptyline, olanzapine, olopatadine, orphenadrine, oxatomide, phenindamine, pheniramine, phenol, pitolisant, pizotifen, promazine, promethazine, propiomazine, quetiapine, quifenadine, ranitidine, risperidone, roxatidine acetate, rupatadine, terfenadine, thonzylamine, trazodone, trimipramine, tripelennamine, triprolidine, tritoqualine, ziprasidone, or zuclopenthixol.

### Anti-Vascular Endothelial Growth Factor (VEGF) Agents and Related Agents

In some embodiments, a dopamine prodrug is combined with an anti-VEGF modulator. In some embodiments, the VEGF modulator may include, but not be limited to VEGF inhibitors vascular endothelial growth factor (VEGF) modulators, e.g., VEGF inhibitors or antagonists, such as tyrosine kinase inhibitors, VEGF specific binding agents, e.g., VEGF antibodies or binding fragments thereof, VEGF binding fusion proteins, and the like; faricimab, bevacizumab, aflibercept, ranibizumab, brolucizumab, and the like; platelet derived growth factor (PDGF) modulators, e.g., PDGF inhibitors or antagonists, such as PDGF specific binding agents, e.g., PDGF antibodies or binding fragments thereof, PDGF binding fusion proteins, and the like; angiopoietin (ANG) modulators, such as ANG2 modulators, e.g., ANG2 inhibitors or antagonists, such as ANG2 specific binding agents, e.g., ANG2 antibodies or binding fragments thereof, ANG2 binding fusion proteins, and the like; placental growth factor (P1GF) modulators, e.g., P1GF inhibitors or antagonists, such as P1GF specific binding agents, e.g., P1GF antibodies or binding fragments thereof, P1GF binding fusion proteins, and the like; tissue necrosis factor (TNF) modulators, such as anti-TNF alpha agents such as antibodies to TNF-a, antibody fragments to TNF-α and TNF binding fusion proteins including infliximab, etanercept, adalimumab, certolizumab and golimumab; mTOR inhibitors such as sirolimus, sirolimus analogues, Everolimus, Temsirolimus and mTOR kinase inhibitors; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents or intraocular pressure lowering agents, such as prostaglandin analogs, ROK inhibitors, beta blockers, alpha agonists, and carbonic anhydrase inhibitors; multi-specific modulators, e.g., bispecific modulators, such as bispecific binding agents, e.g., bispecific antibodies or binding fragments thereof, including agents that specifically bind to both VEGF and ANG2; antibody biopolymer conjugates (including, but not limited to KSI-301, KSI-501, KSI-601) drug eluting microparticles such as poly-lactide-co-glycolide acid (PLGA). In some embodiments, the dopamine prodrug is combined with a complement inhibitor for the treatment of geographic atrophy. In some embodiments, the dopamine prodrug may be combined with 2 or more of the agents listed in this specification. In some embodiments, the dopamine prodrug may be combined with a steroid, including, but not limited to ocular steroids : such as cortisone, dexamethasone, fluocinolone, loteprednol, difluprednate, fluorometholone, prednisolone, medrysone, triamcinolone, betamethasone, fluazacort, hydrocortisone, and rimexolone, and derivatives thereof.

### Excipients

Exemplary pharmaceutically acceptable excipients of the disclosure include those found in Remington: The Science and Practice of Pharmacy, Twenty Second Ed. (London, UK: Pharmaceutical Press, 2013) incorporated herein by reference for such disclosure.

### Surfactants

In some embodiments, the pharmaceutical composition further comprises a surfactant to disburse insoluble ingredients or to improve solubilization. Numerous ocular surfactants are disclosed in US 20190328773, which is incorporated herein by reference. Several of those are listed below. Additional surfactants and relevant ranges are listed in US20180221407A1, which is incorporated herein by reference.

The composition can further include one or more co-solubilizers such as a surfactant. The surfactant may vary, and may include any compound that is surface active or can form micelles. A surfactant may be used for assisting in dissolving an excipient or an active agent, dispersing a solid or liquid in a composition, enhancing wetting, modifying drop size, stabilizing an emulsion, or a number of other purposes. Examples of surfactants may include, but are not limited to, surfactants of the following classes: alcohols, for example polyvinyl alcohol; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal & veg.); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants such as Soluplus® (from BASF); propoxylated & ethoxylated fatty acids, alcohols, or alkyl phenols; protein-based surfactants; sarcosine derivatives; sorbitan derivatives; sucrose and glucose esters and derivatives; and saponins. In some embodiments, the surfactant may include polyethylene glycol (15)-hydroxystearate (CAS Number 70142-34-6, available as SOLUTOL HS 15® from BASF), a polyoxyethylene-polyoxypropylene block copolymer (CAS No. 9003-11-6, available as PLURONIC® F-68 from BASF), polyoxyethylene 40 stearate (P0E40 stearate), polysorbate 80 or polyoxyethylene (80) sorbitan monooleate (CAS No. 9005-65-6), sorbitan monostearate (CAS No. 1338-41-6, available as SPANTM 60 from Croda International PLC), or polyoxyethyleneglyceroltriricinoleate 35 (CAS No. 61791-12-6, available as CREMOPHOR EL® from BASF), ethoxylated castor oil, such as Cremophor EL (CAS Number 61791-12-6). Suitable co-solubilizers include, but are not limited to, povidone, and acrylates (e.g. PEMULEN®).

In some implementations, the surfactant is a non-ionic surfactant that can in some instances include polyoxyethylene sorbitan monooleate (Polysorbate-80) represented by CAS No. 9005-65-6, such as Tween® 80, available from Sigma-Aldrich. In some implementations, the non-ionic surfactant includes polyoxyethylene lauryl ether represented by CAS No. 9002-92-0, such as Brij® 35, available from Sigma-Aldrich. In some implementations, the non-ionic surfactant polyol includes poly(ethylene glycol)-block-poly(propylene glycol)-block-poly(ethylene glycol) represented by CAS No. 9003-11-6, such as Pluronic™ F-127, available from BASF SE. Other non-ionic surfactants are considered herein including, but not limited to ethoxylates, fatty alcohol ethoxylates, alkylphenol ethoxylates, fatty acid ethoxylates, special ethoxylated fatty esters and oils, ethoxylated amines and/or fatty acid amides, terminally blocked ethoxylates, fatty acid esters of polyhydroxy compounds, fatty acid esters of glycerol, fatty acid esters of sorbitol, Tweens, fatty acid esters of sucrose, alkyl polyglucosides, amine oxides, sulfoxides, phosphine oxides.

The amount of surfactant may vary. In some implementations, the surfactant can be used at a concentration from about 0.05% w/v to about 5.0% w/v, preferably 0.05% w/v to about 0.5% w/vv. Some preferred concentrations of the surfactant include 0.04% w/v, 0.045% w/v, 0.05% w/v, 0.055% w/v, 0.06% w/v, 0.065% w/v, 0.07% w/v, 0.075% w/v, 0.08% w/v, 0.085% w/v, 0.09% w/v, 0.095% w/v, 0.10% w/v, 0.15% w/v, 0.20% w/v, 0.25% w/v, 0.30% w/v, 0.35% w/v, 0.40% w/v, 0.45% w/v, 0.50% w/v, 0.55% w/v, 0.60% w/v, 0.65% w/v, 0.70% w/v, 0.75% w/v, 0.80% w/v, 0.85% w/v, 0.90% w/v, 0.95% w/v, 1.0% w/v, 1.5% w/v, 2.0% w/v, 2.5% w/v, 3.0% w/v, 3.5% w/v, 4.0% w/v, 4.5% w/v, and 5.0% w/v and may all be used in conjunction with the implementations described herein.

The surfactant incorporated in the compositions is not limited by class. Various classes of surfactants can be incorporated including, but not limited to anionic, cationic, zwitterionic, and nonionic surfactants. It should also be appreciated that multiple combinations of surfactants can be included.

### Chelating Agents

In some embodiments, the pharmaceutical composition further comprises a chelating agent. Potential chelating agents include, but are not limited to the following: EDTA, disodium edetate, tetrasodium edetate. In some embodiments, the chelating agent is present in the pharmaceutical composition in an amount of about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% (w/w). In some embodiments, the chelating agent is EDTA and is present in the pharmaceutical composition in an amount of about 0.05% (w/w). In some embodiments, the chelating agent is disodium edetate and is present in the pharmaceutical composition in an amount of about 0.05% (w/w). US20180221407A1, which discloses chelating agents, is incorporated herein by reference.

### Buffers

In some embodiments, the pharmaceutical composition further comprises a buffer to maintain the pH in an acceptable range, for example pH of 2 to 14, including 5 to 8. In some embodiments the buffer is used to maintain the pH at about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.0, about 8.1, about 8.2, about 8.3, about 8.4, about 8.5, about 8.6, about 8.7, about 8.8, about 8.9, or about 9.0. In some embodiments the buffer is used to maintain the pH between about 7.0 and about 8.0. In some embodiments the buffer is used to maintain the pH between about 7.0 and about 7.4. In some embodiments the buffer is used to maintain the pH between about 6.5 and about 8.0. In some embodiments the buffer is used to maintain the pH between about 6.8 and about 7.8. In some embodiments the buffer is used to maintain the pH near the pH of human tears (pH 7.4).

In some embodiments, suitable buffering agents include, but are not limited to phosphate buffers, such as sodium phosphate monobasic and sodium phosphate dibasic. In some embodiments, suitable buffers include but are not limited to citrate buffer, acetate buffer, sulfonate buffer, borate buffer, and any combinations thereof.

In some embodiments, the boric acid buffer is a 1.9% solution of boric acid in purified water or sterile water. In some embodiments, the boric acid solution is in a range from 0.01% to 25%. In some embodiments, the boric acid solution is in a range from 0.1% to 3%. In some embodiments, the boric acid solution is in a range from 1% to 2%. In some embodiments, the boric acid solution is in a range from 1.5% to 2.5%.

In some embodiments, Sorensen's modified phosphate buffer can be used to attain a pH value between about 6.5 to 8.0. In some embodiments, Sorensen's modified buffer comprises two stock solutions, one acidic comprising NaH2PO4 and one basic comprising Na2HPO4. In some embodiments, sodium chloride is added to achieve isotonicity. Several formulations are described in the table.

TABLE
Sorensen's Modified Phosphate Buffer

| | mL of Na2HPO4 | | NaCl required for |
|---|---|---|---|
| mL of NaH2PO4* | solution** | pH | isotonicity (g/100mL) |
| 90 | 10 | 5.9 | 0.52 |
| 80 | 20 | 6.2 | 0.51 |
| 70 | 30 | 6.5 | 0.5 |
| 60 | 40 | 6.6 | 0.49 |
| 50 | 50 | 6.8 | 0.48 |
| 40 | 60 | 7 | 0.46 |
| 30 | 70 | 7.2 | 0.45 |
| 20 | 80 | 7.4 | 0.44 |
| 10 | 90 | 7.7 | 0.43 |
| 5 | 95 | 8 | 0.42 |

*8.006 g NaH2PO4/1000 mL with Purified Water
**9.473 gNa2HPO4/1000 mL with Purified Water

### Tonicity Agents

In some embodiments, the pharmaceutical composition includes a tonicity agent, including but not limited to, sodium chloride, sodium nitrate, sodium sulfate, glycerol, mannitol, sucrose, dextrose, sorbitol, and mixtures of the same. In some embodiments, an ophthalmic formulation has an osmolality (measure of tonicity) of at least about 200 mOsmol/kg, with a range of 200 to 500 mOsmol/kg, including 200 to 350 mOsmol/kg and 400 mOsmol/kg. In one embodiment, the formulation is designed to have an osmolality substantially equivalent to that in a biological eye, including a human eye.

Additional tonicity agents and relevant ranges are listed in US20180221407A1, which is incorporated herein by reference.

In some embodiments, the pharmaceutical composition comprises a tonicity agent in an amount of about 0.001%, about 0.0015%, about 0.002%, about 0.0025%, about 0.003%, about 0.0035%, about 0.004%, about 0.0045%, about 0.005%, about 0.0055%, about 0.006%, about 0.0065%, about 0.007%, about 0.0075%, about 0.008%, about 0.0085%, about 0.009%, about 0.0095%, about 0.01%, about 0.015%, about 0.02%, about 0.025%, about 0.03%, about 0.035%, about 0.04%, about 0.045%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, or about 2% (w/w).

In some embodiments, the pH of the pharmaceutical composition is modified via use of a suitable base. Suitable bases include, but are not limited to sodium hydroxide, potassium hydroxide, barium hydroxide, and/or combinations of the same.

### Solvents/Vehicles/Diluents

In some embodiments, the pharmaceutical composition comprises a solvent including, but not limited to water for injection, ringer's solution, or normal saline solution. Other vehicles may be used, including, but not limited to water soluble polyethers such as polyethylene glycol, glycerin, polyvinyls such as polyvinyl alcohol and povidone, cellulose derivatives such as methylcellulose and hydroxypropyl methylcellulose, petroleum derivatives such as white petrolatum, polymers of acrylic acids such as carboxypolymethylene gel, vegetable fats and polysaccharides such as dextrans, and glycosaminoglycans such as sodium hyaluronate and/or combinations of the same.

### Antioxidants

In some embodiments, the pharmaceutical composition comprises an antioxidant to protect against oxidation. Potential antioxidants include, but are not limited to, sodium bisulfite (0.01% to 1%), sodium metabisulfite (0.01% to 1%), thiourea (0.01% to 1%), and disodium edetate (0.01% to 1%). Additional antioxidants include, but are not limited to sodium thiosulfate, acetone sodium bisulfite, gentisic acid, gentisic acid ethanolamide, sodium formaldehyde sulfoxylate, thiourea, butylated hydroxyanisole, butylated hydroxytoluene, esters of gallic acid ascorbic acid, salts of ascorbic acids including ascorbyl palmitate, sodium ascorbate, retinoids and derivatives of Vitamin A, acetylcysteine, thioglycerol, Vitamin E and its derivatives or combinations thereof. Additional antioxidants are listed in WO2019171260A1, which is incorporated herein by reference.

### Preservatives

In some embodiments, preservatives are necessary to enable extended half-life of the product. Suitable preservatives include, but are not limited to, benzalkonium chloride, sodium chlorite, benethonium chloride, purite, methylparaben, propylparaben, ethyl paraben, butyl paraben, perborates, phenol and its derivatives, benzyl alcohol, chlorobutanol, polyquad and combinations thereof. Sodium chlorite includes stabilized chlorine dioxide, commercially available as Purite, which is an aqueous solution of sodium chlorite (NaClO2). U.S. Pat. No. 5,424,078 is incorporated herein by reference in its entirety, discloses the use of stabilized chlorine dioxide as a preservative. In some embodiments, stabilized chlorine dioxide can be used as a preservative for ophthalmic formulations, and is present in an amount ranging from 0.0002 to about 0.2 weight/volume percent, including 0.0002 to 0.02 weight/volume percent. In some embodiments, stabilized chlorine dioxide is present in an amount ranging from 0.004 to about 0.01.

In some embodiments, a preservative is not used.

### Viscosity Agents

In some embodiments, viscosity enhancers are used to increase the contact time of the drug with the target tissue. In some embodiments, the viscosity agent includes, but is not limited to hydroxyethyl cellulose, hydroxypropyl methylcellulose, hydroxymethyl cellulose, polyvinyl pyrrolidone, polyvinyl alcohol, saccharide such as lactose, mannitol, maltose, hyaluronic acid or its salt such as sodium hyaluronate, potassium hyaluronate, chondroitin sulfate, carboxyvinyl polymer, cross linked polyacrylate, sodium alginate, gum tragacanth, chitosan, and mixtures thereof. In some embodiments, viscosity enhancers include hydrogels, including polyelectrolye complexes.

A viscosity enhancer may comprise an acrylic acid or acrylate polymer, either cross-linked or non-cross-linked such as polycarbophil, for example CARBOPOL® (B.F. Goodrich, Cleveland, Ohio) and CARBOPOL 980®. Other commercially available thickeners may include HYPAN® (Kingston Technologies, Dayton, N.J.), NATROSOL® (Aqualon, Wilmington, Del.), KLUCEL® (Aqualon, Wilmington, Del.), or STABILEZE® (ISP Technologies, Wayne, N.J.). Other useful gelling polymers may include carboxymethyl cellulose, hydroxyethyl cellulose, hydroxypropylcellulose, cellulose gum, MVA/MA copolymers, MVE/MA decadiene crosspolymer, PVM/MA copolymer, etc.

In some instances, the viscosity of the formulation ranges from 0.005 to 5000 centipoise, such as 1 to 500 centipoise and including 0.1 to 200 centipoise and 0.5 to 40 centipoise, including 2 to 25 centipoise. In some formulations, the viscosity ranges from 12 to 25 centipoise, including 12 to 20 centipoise and 15 to 18 centipoise.

Additional viscosity agents and relevant ranges are listed in US20180221407A1, which is incorporated herein by reference.

### Extended Release

In some embodiments, the formulation may incorporate agents designed to deliver a therapeutic agent over an extended period of time. These include polyactide-co-glycolide (PLGA) nanoparticles. In some embodiments, Chitosan is used to prolong drug delivery. Chitosan is a polymer obtained by Alkaline or enzymatic deacetylation of chitin. Chitozan consists of repeating units of N-acetly-D-glucosamine and D-glucosamine linkined by beta-(1-4) glycosidic bonds (Figure). (Irimia T, et al. Strategies for improving ocular drug bioavailability and corneal wound healing with chitosan-based delivery systems. Polymers. 2018)

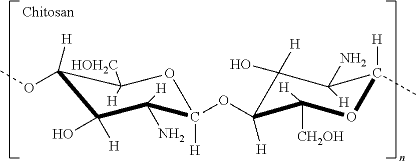

### Mucoadhesive Agents

Mucoadhesive delivery systems help improve drug absorption for therapeutic agents delivered to mucous membranes. These delivery systems work through the formation of adhesions to the mucosal surface, facilitating controlled release of a drug to the target tissue. Mucoadhesive agents include, but are not limited to, natural mucoadhesive polymers. These include, but are not limited to, chitosan, chitosan derivatives including thiolated chitosan derivatives, alginate including sodium alginate, human serum albumin, gellan gum, xanthan gum, guar gum, sodium hyaluronate, tamarind gum polysaccharide, carrageenan, xyloglucan, arabino-galatan, carbomer, cellulose derivatives, poloxamer, eudragit, and poly (D,L-lactide-co-glycolide). In some embodiments, chitosan-based nanoparticle systems are also used to enhance drug delivery in part through sustained drug release at the ocular surface.

In some embodiments, mucoadhesive agents include, but are not limited to, synthetic mucoadhesive polymers. These include, but are not limited to poly (acrylic acid) or carbomer, eudragit, cellulose derivatives, poly (D,L-lactic acide), poly (D,L-lactide-co-glycolide), poly (lactic-co-glycolic acid) (PLGA), PEG, PLGA-PEG suspensions, and poloxamers. Additional mucoadhesive delivery systems are described by Khare et al, which is herein incorporated in its entirety by reference (Khar A et al. Mucoadhesive polymers for enhancing retention in ocular drug delivery: a critical review. Rev Adhesion Adhesives, 2014).

### Terminal Sterilization

In some embodiments, the ophthalmic solution is filled in a non-sterile environment, for example, an ISO class 7 room, and terminally sterilized. Acceptable forms of terminal sterilization include, but are not limited to, electron-beam (E-beam), gamma radiation, autoclaving and ethylene oxide. In some embodiments, e-beam terminal sterilization is utilized. In some embodiments, the ophthalmic composition is exposed to 1 to 100 kGy, including 5 to 50 kGy. In some embodiments, the ophthalmic composition is exposed to 10-35 kGy, including 15-25 kGy. In some embodiments, the ophthalmic composition is cooled immediately prior to and during terminal sterilization. In some embodiments, the ophthalmic composition is frozen immediately prior to and during terminal sterilization.

In some embodiments, the ophthalmic formulation is filled aseptically, and no terminal sterilization is needed. In some embodiments, filtration through an appropriate membrane is utilized to achieve target bioburden levels.

### Dosage Forms

The dosage form of the pharmaceutical composition will, in part, be determined by the mode of administration. The invention may be delivered in one or several of the following manners: topical, subconjunctival injection, punctal plug, Schlemm's canal device, scleral depot, intravitreal injection, suprachoroidal injection, subtenons injection, topical or injectable suspension, lyophilized agent+buffer, ointment, emulsion, contact lens, IOL depot, biodegradable polymer, corneal implant. Additionally, inhalation, injectable fluids, and oral forms may be employed. Topical preparations may include eye drops, ointments, sprays and patches. Inhalation preparations may include liquid formulations, mists, and sprays. Oral formulations may include liquids or solids. Liquids may include syrups, solutions or suspensions. Solid formulations may include powders, pills, tablets or capsules. For solids, conventional non-toxic solid carriers can include pharmaceutical grades of mannitol, lactose, starch, or magnesium stearate.

In some embodiments, the pharmaceutical composition is formulated as an ophthalmic solution. Ophthalmic solutions are sterile, and are intended for ocular installation. There are several requirements for ophthalmic solutions, relating to pH, osmolarity, buffering, viscosity, and packaging requirements. Ophthalmic solutions can be formulated as a liquid and a powder, or multiple liquids, with combination immediately before use. Alternatively, all components can be formulated as a single solution. Viscosity can be modified as discussed elsewhere in this specification, with the goal of increasing contact time of the solution with the ocular surface.

In some embodiments, the pharmaceutical composition is formulated as an ophthalmic suspension. Suspensions can be designed to increase ocular contact time. In some embodiments, the ophthalmic suspension consists of active ingredient(s) particle size that is optimized to result in extended release in the conjunctival fornice, with avoidance of foreign body sensation in the eye. In some embodiments, the particle size is in a range from 0.005 to 20 micrometers, including 1 to 10 um. In some embodiments, the formulation is designed to prevent aggregation of particles, reducing the risk of ocular foreign body sensation.

In some embodiments, the ophthalmic composition is formulated as an ointment. In some embodiments, the ophthalmic composition is formulated as a gel. In some embodiments, the ophthalmic composition is formulated as an emulsion.

In some embodiments, the composition described herein may comprise a protein, including, but not limited to a soluble receptor, an antibody fragment, antigen binding fragment, or peptide. In some embodiments, the antibody is a humanized antibody. In some embodiments, the antibody is a fully human antibody

### Stability

In some embodiments, stability refers to a pharmaceutical composition having a low number of impurities, for example, less than 5% w/w total impurities following a storage period. In some embodiments, the pharmaceutical composition has about 5% w/w, about 4% w/w, about 3% w/w, about 2.5% w/w, about 2% w/w, about 1.5% w/w, about 1% w/w, about 0.5% w/w, or about 0.25% w/w total impurities at the end of a given storage period.

In some embodiments, the pharmaceutical composition is stable at refrigerated conditions for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 27 months, at least 30 months, at least 33 months, at least 36 months, or at least 48 months or any interval time period.

In some embodiments, the pharmaceutical composition is stable at room temperature conditions for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, at least 24 months, at least 27 months, at least 30 months, at least 33 months, at least 36 months, or at least 48 months or any interval time period.

In some embodiments, the pharmaceutical composition is stable at accelerated aging conditions for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, or at least 18 months or any interval time period.

### Delivery Device

The invention may delivered by one or several of the following: a standard container closure, blow fill seal/single use, multi-part container closure, including 2-part container closures, dual chamber syringe, injector for biodegradable implant, nanoparticles, drug eluting scleral-fixated implant, and piezo-print delivery technology/microdosing devices.

### Method of Use

Also disclosed herein are methods of treating or preventing an eye condition in a subject, the method comprising administering to the subject a pharmaceutical composition described herein.

In some embodiments of a method of preventing or reversing axial elongation in a subject in need thereof, the pharmaceutical composition comprises: (1) a prodrug that, upon passage through the ocular surface, is converted to a dopamine agonist, and (2) one or more dopamine antagonists. In some embodiments, the pharmaceutical composition comprises: (1) a prodrug such as etilevodopa, that, upon passage through the ocular surface, is converted to a dopamine agonist, and (2) a vesicular monoamine transport inhibitor. In some embodiments, the pharmaceutical composition comprises: (1) a prodrug such as etilevodopa, that, upon passage through the ocular surface, is converted to a dopamine agonist, and (2) a dopamine reuptake inhibitor. In some embodiments, the pharmaceutical composition comprises: (1) etilevodopa, and (2) boronic acid. In some embodiments, the pharmaceutical composition comprises etilevodopa. In some embodiments, the pharmaceutical composition comprises etilevodopa hydrochloride.

In some embodiments, the pharmaceutical composition comprises: (1) docarpamine, and (2) boronic acid. In some embodiments, the pharmaceutical composition comprises: (1) adrogolide, and (2) boronic acid.

### Myopia Treatment

In one embodiment, the ophthalmic composition will be used for the treatment of myopia, including, but not limited to, refractive myopia, mild myopia, moderate myopia, high myopia, progressive myopia, pathologic myopia, degenerative myopia, myopia secondary to form deprivation, lens induced myopia, myopia secondary to hyperopic defocus, myopia secondary to flickering light, myopia secondary to monochromatic light, myopia secondary to axial elongation, myopia secondary to corneal elongation, lenticular myopia, and myopia associated with syndromes including but not limited to Acromelic frontonasal dysostosis, Alagille syndrome, Alport syndrome, Angelman syndrome, Bardet-Biedl syndrome, Beals syndrome, Beaulieu-Boycott-Innes, syndrome, Bohring-Opitz syndrome, Bone fragility and, contractures, arterial rupture and deafness, Branchiooculofacial syndrome, Cardiofaciocutaneous syndrome, Cohen syndrome, Cornelia de Lange, syndrome, Cowden syndrome, Cranioectodermal dysplasia, Cutis laxa, Danon disease, Deafness and myopia, Desanto-Shinawi syndrome, Desbuquois dysplasia, Donnai-Barrow syndrome, DOORS, Ehlers-Danlos syndrome, Macrocephaly/megalencephaly syndrome, Marfan syndrome, Marshall syndrome, Microcephaly with/without chorioretinopathy; lymphedema; and/or mental retardation, Mohr-Tranebjaerg syndrome, Mucolipidosis, Muscular dystrophy, Nephrotic syndrome, Noonan syndrome, Oculocutaneous albinism, Oculodentodigital dysplasia, Pallister-Killian syndrome, Papillorenal syndrome, Peters-plus syndrome, Pitt-Hopkins syndrome, Pontocerebellar hypoplasia, Poretti-Boltshauser syndrome, Prader-Willi syndrome, Pseudoxanthoma elasticum, Renal hypomagnesemia, SADDAN, Schaaf-Yang syndrome, Schimke immunoosseous, dysplasia, Schuurs-Hoeijmakers syndrome, Schwartz-Jampel syndrome, Sengers syndrome, Short stature; hearing loss; retinitis pigmentosa and distinctive facies, Short stature; optic nerve atrophy; and Pelger-Huet anomaly, SHORT syndrome, Short-rib thoracic dysplasia with/without, polydactyly, Shprintzen-Goldberg syndrome, Singleton-Merten, syndrome, Small vessel brain disease with/without ocular anomalies, Smith-Magenis, syndrome, Spastic paraplegia and psychomotor retardation with or without seizures, Split hand/foot, malformation, Stickler syndrome, Syndromic mental retardation, Syndromic microphthalmia, Temtamy syndrome, White-Sutton syndrome, Zimmermann-Laband syndrome, Achromatopsia, Aland Island eye disease, Anterior segment dysgenesis, Bietti crystalline corneoretinal dystrophy, Blue cone monochromacy, Brittle cornea syndrome, Cataract, Colobomatous macrophthalmia with microcornea, Cone dystrophy, Cone rod dystrophy, Congenital microcoria, Congenital stationary night blindness, Ectopia lentis et pupillae, High myopia with cataract and vitreoretinal degeneration, Keratoconus, Leber congenital amaurosis, Microcornea, myopic chorioretinal atrophy, and telecanthus, Microspherophakia and/or megalocornea, with ectopia lentis and/or secondary glaucoma, Ocular albinism, Primary open angle glaucoma, Retinal cone dystrophy, Retinal dystrophy, Retinitis pigmentosa, Sveinsson chorioretinal atrophy, Vitreoretinopathy, Wagner vitreoretinopathy, and Weill-Marchesani syndrome.

The ophthalmic composition may be used in combination with other methods that may reduce or increase axial length. The ophthalmic composition may be used in combination with other methods for preventing or reversing axial elongation including, but not limited to bifocals, progressive lenses, orthokeratology, peripheral blur glasses, and/or peripheral blur contact lenses. The ophthalmic composition may also be used in combination with other pharmacologic treatments for myopia, including Atropine drops.

### Other Uses:

The ophthalmic composition may be used to treat any ophthalmic pathologic condition, including, but not limited to those that result in decreased retinal sensitivity. Dopamine is a critical retinal neuromodulator, and supplementing dopamine may provide therapeutic benefit for subjects with retinal diseases, including, but not limited: exudative age-related macular degeneration, non-exudative age-related macular degeneration, polypoidal choroidal vasculopathy, non-proliferative diabetic retinopathy, proliferative diabetic retinopathy, diabetic macula edema, central retinal vein occlusion, branch retinal vein occlusion, paracentral acute middle maculopathy, peripheral exudative hemorrhagic chorioretinopathy, lipemia retinalis, central serous chorioretinopathy, bilateral diffuse uveal melanocytic proliferation, uveal effusion syndrome, juxtafoveal retinal telangiectasia, myopic maculopathy, choroidal neovascular membrane, ocular histoplasmosis, rhegmatogenous retinal detachment, serous retinal detachment, exudative retinal detachment, tractional retinal detachment, proliferative vitreoretinopathy, retinoschisis, branch retinal artery occlusion, central retinal artery occlusion, combined retinal artery and retinal vein occlusion, ophthalmic artery occlusion, ocular ischemic syndrome, hypertensive retinopathy, retinal microaneurysm, Susac syndrome, retinitis pigmentosa, macular dystrophies including but not limited to Stargardt disease, Stargardt-like dominant macular dystrophy, pattern dystrophy, adult-onset foveomacular vitelliform dysytophy, Sjogren reticular dystrophy of the retinal pigment epithelium (RPE), central areolar choroidal dystrophy, Sorsby macular dystrophy, autosomal dominant radial drusen, north carolina macular dystrophy, complement mediated disease, late onset retinal macular degeneration, occult macular dystrophy, pigmented paravenous retinochoroidal atrophy, Best macular dystrophy, central areolar choroidal dystrophy, unilateral retinal pigment epithelium dystrophy, choroideremia, gyrate atrophy, achromatopsia, autosomal dominant neovascular inflammatory vitreoretinopathy, congenital color deficiency, congenital night blindness disorders, deuteranomaly, dominant cone dystrophy, x-linked cone dystrophy, x-linked retinoschisis, abetalipoprotenemia, Alstrom syndrome, Aicardi syndrome, Cockayne syndrome, Coats disease, Duchenne muscular dystrophy, incontinentia pigmenti, Jalili syndrome, Jeune syndrome, Knobloch syndrome, Marshall syndrome, neurogenic muscle weakness, ataxia, and retinitis pigmentosa (NARP), mitochondrial encephalopmyopathy, lactic acidosis and stroke (MELAS), maternally inherited diabetes and deafness (MIDD), Norrie disease, neurodegeneration with brain iron accumulation/hallervorden-spatz disease (NBIA), neuronal ceroid lipofuscinoses, Batten disease, Senior-Loken syndrome, Sjogren-Larsson syndrome, Usher syndrome, Kerns-Sayer syndrome, primary hyperoxaluria, retinopathy secondary to sickle cell disease, retinopathy secondary to hemoglobinopathies, radiation retinopathy, retinopathy of prematurity, familial exudative vitreoretinopathy, persistent fetal vasculature, white dot syndromes including acute idiopathic maculopathy, acute macular neuroretinopathy, acue posterior multifocal placoid pigment epitheliopathy, acute retinitis pigment epitheliitis, acute zonal occult outer retinopathy, birdshot uveitis, multiple evanescent white dot syndrome, multifocal choroiditis, punctate inner choroiditis, persistent placoid maculopathy, relentless placoid chorioretinopathy, serpiginous choroidopathy, and subretinal fibrosis and uveitis syndrome, hypotony, hypotony maculopathy, autoimmune retinopathies and paraneoplastic retinopathies including cancer associated retinopathy and melanoma associated retinopathy, uveitis including all etiologies of anterior, intermediate, posterior and panuveitis, endophthalmitis, Eales disease, Vogt-Koyanagi-Harada disease, solar retinopathy, welders maculopathy, Terson syndrome, Purtscher retinopathy, shaken baby syndrome, whiplash retinopathy, Valsalva retinopathy, toxic retinopathy including but not limited to toxicity secondary to thioridazine, chloroquine, chlorpromazine, hydroxychloroquine, quinine sulfate, clofazimone, dideoxyinosine, deferoxamine, ergot alkaloids, cisplatin, and carmustine (BCNU), alkylyl nitrates, aminoglycoside antibiotics, bisphosphonates, canthaxanthine, checkpoint inhibitors, epidermal growth factor receptor kinase inhibitors, ocriplasmin, glitazones, methoxyflurane, mitogen-activated protein kinase inhibitors, nitrofurantoin, nucleoside reverse transcriptase inhibitors, pentosan polysulfate, procainamide, talc, tamoxifen, taxanes, methanol, rifabutin, ritonavir, cidofovir, latanoprost, sildenafil, tadalafil, vardenafil, vigabatrin, and interferon; drugs that can result in acute myopia, including but not limited to sulfa antibiotics, acetazolamide, chlorthalidone, disothiazide, ethoxyzolamide, dydrochlorothiazide, metronidazole, sulphonamide, topiramate, and triamterene.

The ophthalmic composition may be used to treat any ophthalmic pathologic condition, including, but not limited to those that result in damage to the optic nerve and nerve fiber layer, including glaucoma, ocular hypertension, primary open angle glaucoma, angle closure glaucoma, anterior ischemic optic neuropathy (AION), non-arteritic anterior ischemic optic neuropathy (NAION).

### Dosing

In some embodiments, the ophthalmic composition is applied to the target tissue daily, twice daily (BID), three times daily (TID), four times daily (QID), hourly, weekly, bi-weekly, every second, third, fourth, fifth, sixth day, every 3 rd week, monthly, every 6th, 7th, 8th, 9th, 10th, 11th, or 12th week, every 4th, 5th, 6th, 7th, 8th, 9th, 10th, 11th, or 12th month, every 18, 24, 30, or 36 months. In some embodiments, the dosing regimen can be modified for a specific patient or subject based on any number of factors, including, but not limited to, the subjects age, weight, degree of axial elongation, rate of axial elongation, family history of disease, or genetic testing.

In one embodiment, the ophthalmic composition is designed for application at night (qhs), thereby minimizing potential ocular discomfort due to transient dilation and any potential loss of ocular accommodation. Administering at night, including shortly before bed, can help reduce the potential side effect of slightly blurred vision that can occur with eye drop instillation, including viscous ophthalmic compositions.

In one embodiment, the ophthalmic composition may be delivered frequently (loading dose), including but not limited to hourly, for an initial period of several days to weeks, followed by a decreased treatment interval.

In one embodiment, the ophthalmic composition may be delivered via a single intravitreal injection or multiple intravitreal injections. In one embodiment, the ophthalmic composition may be delivered via an intravitreal injection for a loading dose, followed by topical therapy.
1. Pharmaceutical composition comprising a prodrug that, upon passage through the ocular surface, is converted to a dopamine agonist.
2. Pharmaceutical composition according to clause 1 that further comprises a binding agent that neutralizes activated drug that is converted prior to delivery to the target tissue.
3. Pharmaceutical composition comprising a prodrug that, upon passage through the ocular surface, is converted to a dopamine agonist for the treatment of myopia.
4. Pharmaceutical composition of clause 1, wherein the prodrug is converted to a selective dopamine agonist.
5. Pharmaceutical composition of clause 2, wherein the prodrug is converted to a selective D1 agonist.
6. Pharmaceutical composition of clause 2, wherein the prodrug is converted to a selective D2 agonist.
7. Pharmaceutical composition of clause 1, wherein the prodrug is converted to a non-selective dopamine agonist.
8. Pharmaceutical composition comprising a prodrug that, upon passage through the ocular surface, is converted to a dopamine agonist and (2) one or more dopamine antagonists.
9. Pharmaceutical composition comprising a prodrug that, upon passage through the ocular surface, is converted to a dopamine agonist and (2) one or more dopamine reuptake inhibitors.
10. Pharmaceutical composition comprising a prodrug that, upon passage through the ocular surface, is converted to a dopamine agonist and (2) one or more vesicular monoamine transport inhibitors.
11. Pharmaceutical composition of clause 8, wherein one or more of the dopamine antagonists comprises a prodrug that, upon application to or passage through the ocular surface, is converted to a dopamine antagonist.
12. Pharmaceutical composition of clause 8, wherein the dopamine antagonist inhibits one or more parts of the intracellular signaling cascade activated by a dopamine receptor.
13. Pharmaceutical composition of clause 8, wherein the dopamine antagonist reduces or blocks activation of a dopamine receptor.
14. Pharmaceutical composition of clause 8, wherein the dopamine antagonist acts by inactivating a dopamine agonist.
15. Pharmaceutical composition of clause 8, wherein one or more of the dopamine antagonists comprises a selective dopamine antagonist.
16. Pharmaceutical composition of clause 8, wherein one or more of the dopamine antagonists comprises a selective D2 antagonist.
17. Pharmaceutical composition of clause 8, wherein one or more of the dopamine antagonists comprises a selective D1 antagonist.
18. Pharmaceutical composition of clause 8, wherein one or more of the dopamine antagonists comprises a non-selective antagonist.
19. Pharmaceutical composition of clause 8, wherein one or more of the dopamine antagonists is unable to penetrate the ocular surface.
20. Pharmaceutical composition of clause 8, wherein one or more of the dopamine antagonists is able to penetrate the ocular surface.
21. Pharmaceutical composition of clause 8, wherein the dopamine antagonists comprises one or more dopamine antagonists that are unable to penetrate the ocular surface and one or more dopamine antagonists that are able to penetrate the ocular surface.
22. Pharmaceutical composition of clause 19, wherein the dopamine antagonist that is able to penetrate the eye comprises a selective dopamine antagonist.
23. Pharmaceutical composition of clause 19, wherein the selective antagonist comprises a prodrug that, upon passage through the ocular surface, is converted to a selective antagonist.
24. Pharmaceutical composition of clause 8, wherein one of the dopamine antagonist comprises a molecule which binds to and inactivates a dopamine agonist.
25. Pharmaceutical composition of clause 1, wherein the molecule comprises a small molecule.
26. Pharmaceutical composition of clause 1, wherein the small molecule comprises a lewis acid.
27. Pharmaceutical composition of clause 24, wherein the lewis acid comprises an organoborane.
28. Pharmaceutical composition of clause 25, wherein the organoborane comprises a boronic acid.
29. Pharmaceutical composition of clause 1, wherein the molecule comprises a peptide.
30. Pharmaceutical composition of clause 1, wherein the molecule comprises a protein.
31. Pharmaceutical composition of clause 26, wherein the protein comprises a soluble receptor.
32. Pharmaceutical composition of clause 26, wherein the protein is an antibody or fragment thereof.
33. Pharmaceutical composition of clause 7, wherein one or more of the dopamine antagonists has poor oral bioavailability.
34. Pharmaceutical composition of clause 7, wherein one or more of the dopamine antagonists is modified to prevent systemic absorption.
35. Pharmaceutical composition of clause 7, wherein one or more of the dopamine antagonists is formulated to prevent systemic absorption.
36. Pharmaceutical composition according to any of the preceding clauses wherein the composition comprises a solution.
37. Pharmaceutical composition according to any of the preceding clauses wherein the composition comprises an ointment.
38. A method of applying the pharmaceutical composition of clauses 1-37, wherein a patient applies a drop onto the eye to inhibit axial elongation.
39. A method of applying the pharmaceutical composition of clauses 1-38, wherein a patient applies a drop onto the eye at a frequency of once per evening to inhibit axial elongation.
40. A method of applying the pharmaceutical composition of clause 1 comprising an intravitreal injection.
41. A method of applying the pharmaceutical composition of clause 1 comprising an intraocular drug delivery device.
42. A method of combining a pharmaceutical agent that increases intraocular dopamine with an agent that reduces degradation of dopamine, resulting in an increased concentration of dopamine in the eye for the purpose of modulating axial elongation.

The following examples are offered by way of illustration and not by way of limitation.

### EXPERIMENTAL

### I. Ocular Penetrance and Safety of the Dopaminergic Prodrug Etilevodopa

### A. Introduction

The prevalence of myopia (“nearsightedness”) is increasing at an alarming pace. In the past 50 years, myopia prevalence in the United States and Europe has doubled and in China has jumped from 20% to 90% of the population.¹ In the next 30 years, high myopia is expected to more than triple to near 10% of the global population.² Although there is substantive economic burden from refractive errors alone, high myopia carries significant visual morbidity including increasing risk of cataract, peripapillary deformation, optic neuropathy, retinal detachment, myopic degeneration, myopic foveoschisis, retinoschisis, macular holes, dome-shaped macula, and choroidal neovascularization—many of which cause irreversible vision loss.³

The critical feature of myopia is axial length elongation of the eye, which is responsible for both the refractive changes and pathologic consequences of myopia. Myopia is caused by excessive axial length elongation. Axial length homeostasis is a complex process that is incompletely understood. Currently evolving theories involve a visual stimuli trigger that is transduced from the retina to the sclera through chemical signals, eventually affecting scleral remodeling.⁴ Neurotransmitters, proteases, and growth factors have all been implicated in this process.⁴ One such regulator is dopamine, a neurotransmitter released from amacrine and interplexiform cells of the retina, which has been shown to affect axial elongation.

While dopamine has not been clinically validated to date, pre-clinical data suggests that it is a critical regulator of axial elongation. Dopaminergic agents (i.e., dopamine, apomorphine, levodopa, ADTN, SKF-38393, quinpirole) appear highly effective at reducing axial length in animal models of myopia. Similarly, dopamine antagonists appear to halt this antimyopic effect (haloperidol, SCH23390, spiperone, sulpiridine, and 6-OHDA). Since initial studies with subconjunctival injections of apomorphine in chicks,5 dopaminergic agents have been shown to inhibit axial elongation in chicks,⁶⁻⁹ rabbits,^{10,11} guinea pigs,^{7,12,13} mice,¹⁴⁻¹⁶ tree shrews,¹⁷ and macaques.¹⁸ Dopaminergics are effective in inhibiting both form deprivation myopia,¹⁹⁻²¹ and lens-induced myopia,^{8,20,22} and have shown efficacy whether delivered systemically via the intraperitoneal route⁷ or locally via subconjunctival,⁵ peribulbar,²³ or intravitreal injection.^{8,10,11,22} Unlike anticholinergic agents currently in use, dopaminergic agents have limited effects on accommodation and, without crossover antimuscarinic activity, have no effect on pupillary dilation.²⁴ In animal testing, dopaminergics are more effective at preventing myopia.²⁵ Whereas even low-dose atropine 0.01% has shown potential toxicity on electroretinography (ERG),^{26,27} dopaminergics have not exhibited any toxicity.²⁵ On the basis of these data, dopaminergic compounds appear to inhibit axial elongation and therefore may be candidates for the treatment of myopia.

A major factor limiting clinical development of dopaminergic compounds for the treatment of myopia has been the method of delivery. Most dopaminergic compounds are either poorly soluble or have limited intraocular penetration when applied topically. Myopia progression for juvenile-onset myopia primarily occurs between the ages of 6 and 21 years of age.²⁸ Frequent delivery of dopaminergic agents via peribulbar, intravitreal, or intraperitoneal injections is not clinically viable. It is therefore necessary to develop topical dopaminergic agents that have high ocular penetration allowing for topical delivery in children with progressive myopia. A potential solution is the use of dopaminergic prodrugs. Levodopa, a dopamine prodrug, has demonstrated efficacy at slowing axially elongation in the chick after topical administration.^{25, 29} However, it is significantly less effective when delivered topically rather than intravitreally. We hypothesized that enhancing both lipophilicity and solubility with esterification would further enhance corneal penetration. Such an approach has the potential to achieve a higher intravitreal dopamine concentration after topical administration. Etilevodopa is an ester prodrug of levodopa and, we hypothesized, would provide for improved intraocular penetration after topical administration. This study sought to assess the ex vivo ocular penetration and in vivo safety of etilevodopa.

### B. Methods

### 1. Ocular Penetration of Dopamine and Dopaminergic Derivatives Ex Vivo

To assess the vitreous penetration, dopamine (dopamine hydrochloride; Sigma-Aldrich, St. Louis, Mo., USA), and etilevodopa (BOC Sciences, Shirley, N.Y., USA) were freshly dissolved in phosphate buffered saline solution (PBS, pH 7.4; Aniara, West Chester, Ohio, USA) at the concentration of 0.5%, whereas levodopa (Sigma-Aldrich) was freshly dissolved in distilled water at 0.5%. Thirty microliters of each solution was placed onto the cornea of enucleated porcine eyes (Animal Technologies, Tyler, Tex., USA; at least six porcine eyes were studied for each treatment), which were no more than 30 hours since enucleation and were maintained on ice throughout experimentation. PBS was used as a vehicle control. The vitreous humor sample was collected after 24 hours for quantitative measurement of dopaminergic derivative level using an enzyme-linked immunosorbent assay (ELISA) kit according to the manufacturers' protocol (dopamine ELISA kit; Immuno-Biological Laboratories, Inc., Minneapolis, Minn., USA). The penetration to both anterior chamber and vitreous of etilevodopa at 10% (this concentration represented the maximum solubility of etilevodopa) was further studied at 0, 0.5, 1, 6, 12, 18, 24, 48, and 72 hours to establish a time-penetration curve (at least three porcine eyes were studied at each time point).

### 2. Animals

The use of rats followed the animal study guidelines of the Association of Research in Vision and Ophthalmology and was approved by the Administrative Panel on Laboratory Animal Care (APLAC) at Stanford University. Both male and female Long Evans rats (six weeks of age; body weight 150 to 200 g; Charles River, Wilmington, Mass., USA) were housed at constant temperatures, with a 12-hour light/dark cycle and food and water available as desired.

### 3. Pharmacokinetics of Topical Etilevodopa in Long Evans Rat Eyes

To assess the short-term pharmacokinetic effect, 5 μL of etilevodopa 10% was instilled onto a rat eye. At one, two, four, eight, and 24 hours, rats were sacrificed (at least three rats were studied at each time point). Serum was obtained by allowing the blood sample to clot at room temperature for 0.5 to 1 hour, followed by centrifuging at 13,000 rpm for 10 minutes (MiniSpin Eppendorf; Thermo Fisher Scientific, Waltham, Mass., USA). Serum was then frozen at −80° C. until tested. Eyes were enucleated and collected for quantitative measurement of dopaminergic derivative levels. Concentrations of dopaminergic derivative in both whole eye and vitreous/retina were measured for pharmacokinetic evaluation. Briefly, tissue samples were incubated with 150 μL lysis buffer (CelLytic MT Cell Lysis Reagent; Sigma-Aldrich) for 0.5 to 1 hour, followed by centrifuge at 13,000 rpm for 10 minutes, and the supernatant was collected. All samples were immediately frozen at −80° C. until tested. The level of dopaminergic derivative was measured in both serum and eye using a dopamine ELISA kit (Immuno-Biological Laboratories, Inc.) according to the manufacturer's instruction.

To assess the long-term pharmacokinetic effect, 5 μL of etilevodopa 10% was administered once daily to a rat eye. At 14 and 28 days, rats were sacrificed, and serum was obtained for measurement of dopaminergic derivative levels (four rats were studied at each time point).

### 4. Clinical Examination, Optical Coherence Tomography (OCT) Imaging and ERG Measurement

Etilevodopa 10% 5 μL was administered as an eye drop once daily to one eye of each rat for 28 consecutive days, and the contralateral eye received PBS as a control. To assess the tolerability after administering etilevodopa over time, clinical examination was conducted at 14 and 28 days during treatment. To assess retinal structure and function, spectral-domain OCT (Spectralis HRA+OCT instrument; Heidelberg Engineering, Heidelberg, Germany) and full-field ERG (D300; Diagnosys LLC, Lowell, Mass., USA) were also performed at 14 and 28 days. Briefly, rats were first anesthetized with intraperitoneal injection of a mixture of ketamine hydrochloride (75 mg/kg; Hospira, Inc., Lake Forest, Ill., USA) and xylazine (5 mg/kg; Bedford Laboratories, Bedford, Ohio, USA). The corneas were topically anesthetized with tetracaine 0.5% (Alcon Laboratories, Inc. Fort Worth, Tex., USA), and the pupils were dilated with tropicamide 1% (Akorn, Inc., Lake Forest, Ill., USA) and phenylephrine hydrochloride 2.5% (Akorn, Inc.).

For clinical examination, 1% methylcellulose and a plastic coverslip were applied to the cornea to enhance visualization. A Zeiss OPMI MDO S5 Microscope (Prescott's, Inc., Monument, Col., USA) with an Excelis camera (ACCU-SCOPE, Inc., Commack, N.Y., USA) was used to digitally record ocular photos.

For OCT, 1% methylcellulose and a coverslip were placed on the anesthetized rat cornea. A commercially available 78-D double aspheric fundus lens (Volk Optical, Inc., Mentor, Ohio, USA) was mounted in front of the OCT and images were taken with proprietary software (Eye Explorer, version 3.2.1.0; Heidelberg Engineering). A raster scan of 19 equally spaced horizontal B-scans centered on the optic nerve was captured.

For full-field ERG, rats were dark-adapted overnight. A gold wire loop was placed on the cornea of both eyes, a reference electrode was placed subcutaneously on the nose, and a ground electrode was placed in the tail. ERG responses were recorded from both eyes simultaneously. The flash intensity was set at six increasing intensities of 0.0001, 0.001, 0.01, 0.1, 1, and 3 cd·s/m2. ERG recordings were averaged over 10 presentations of a single one-millisecond flash with a 10-second interstimulus interval. The A-wave amplitude was measured from the baseline to trough, while the B-wave was measured from the trough to peak.

### 5. Histology and Immunohistochemistry

To assess for inflammation and apoptosis, 5 μL of etilevodopa 10% was administered as a once-daily eye drop to one eye of each rat, and the contralateral eye received PBS as a control. Rat eyes were enucleated at 14 and 28 days after treatment and fixed in 4% paraformaldehyde (VWR, Visalia, Calif., USA) overnight at 4° C., dehydrated in graded series of sucrose and alcohol, frozen, and cut into 12 μm sections onto glass slides.

For inflammation, frozen sections were first fixed in 10% formalin (Sigma-Aldrich) for 20 minutes and washed in water, followed by staining the nuclei with hematoxylin for three minutes and washing in water again. The slides were then dipped in lithium carbonate (bluing agent) for 45 seconds, washed in water, immersed in 95% ethanol (2-3 dips), and stained with eosin for another three minutes. After dehydrating in 95% ethanol for one minute and then in 100% ethanol for two minutes, the slides were mounted with Cytoseal 60 and imaged using Nikon Eclipse E800 microscope (Nikon Corp., Tokyo, Japan).

For apoptosis, sections were stained with cleaved caspase-3 (Cell Signaling, Beverly, Mass., USA). Briefly, sections were first washed with washing buffer (0.1% Triton X-100 in PBS) three times for five minutes each, blocked with 5% bovine serum albumin (Fraction V; Sigma-Aldrich) in washing buffer for one hour, followed by incubating in primary antibody-cleaved caspase-3 (1:200 in washing buffer) for two hours. After washing three times for five minutes each, sections were incubated in secondary antibody (AlexaFluor 488 donkey anti-rabbit IgG; 1:400; Invitrogen, Waltham, Mass., USA) in washing buffer for one hour, followed by washing another three times and mounting with 4′,6-diamidino-2-phenylindole (DAPI)-containing mounting media (Vectashield; Vector Laboratories, Burlingame, Calif., USA). Slides were imaged using Leica DMi8 microscope (Leica Microsystems Inc., Buffalo Grove, Ill., USA). Terminal deoxynucleotidyl transferase dUTP nick end labeling (TUNEL) assay was also performed to further detect apoptosis using TUNEL label mix kit (Sigma-Aldrich). TUNEL reaction mixture was prepared using the nucleotide-labeling mix combined with the TUNEL enzyme (Sigma-Aldrich), and the assay was performed according to the manufacturer's instructions.

### 6. Statistical Analysis

Statistical analyses were performed using Prism 9 (Graphpad Software, San Diego, Calif., USA). All results are expressed as mean±SEM and the differences among groups were assessed with statistical tests noted in the figure legends. A P value <0.05 was considered to be statistically significant.

### C. Results

### 1. Ocular Penetration of Dopamine and Dopaminergic Derivatives Ex Vivo

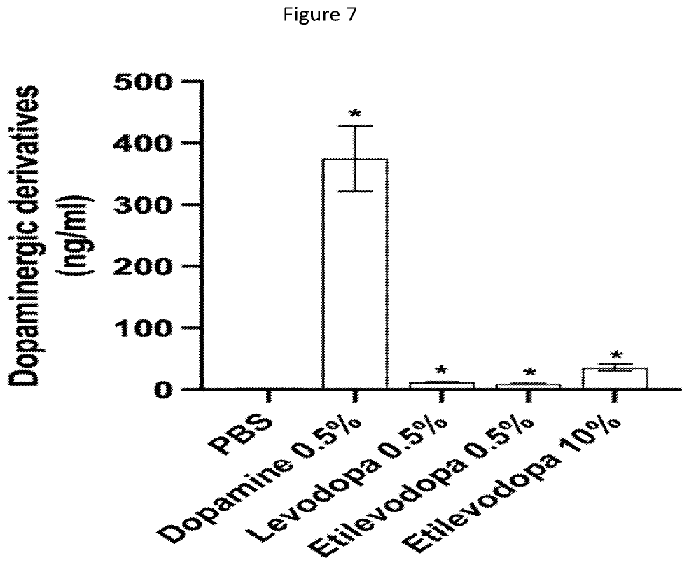

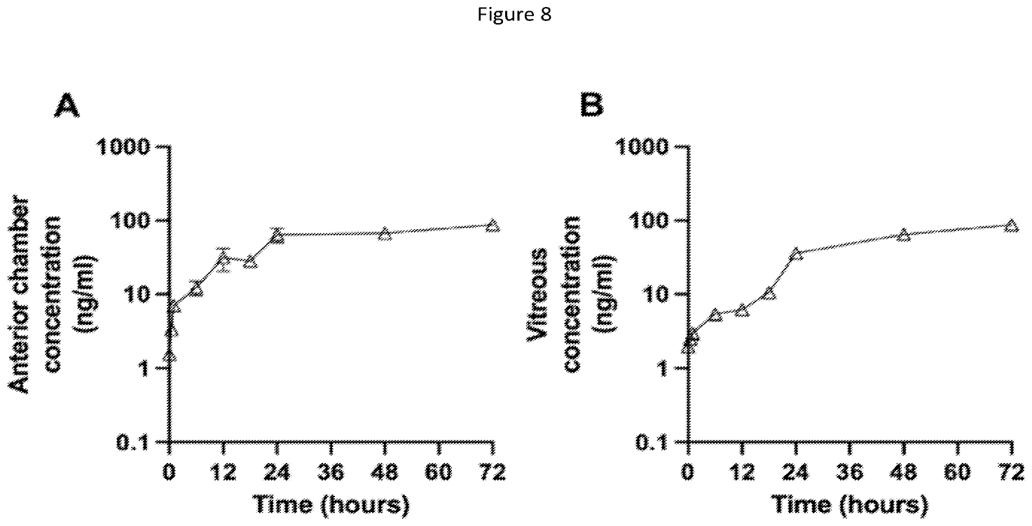

The vitreous penetration of topical dopamine and dopaminergic drugs was assessed using enucleated porcine eyes. As shown in FIG. 7, all studied drugs, that is, dopamine, levodopa and etilevodopa, at 0.5% were able to penetrate the eye and reached the vitreous (FIG. 7) at 24 hours after treatment, with a concentration of 375.1 (P=0.0123), 11.9 (P=0.0028), and 9.7 ng/mL (P=0.0047), respectively. Etilevodopa achieved significant vitreous concentrations when applied as a 10% solution (36.2 ng/mL, P=0.0370). The time-penetration profile of etilevodopa at 10% was further studied over a 72-hour period (FIG. 8). After a single topical administration into a porcine eye, etilevodopa could continuously diffuse into the anterior chamber (FIG. 8, panel A) and vitreous (FIG. 8, panel B), reaching a peak concentration of 87.3 and 87.7 ng/mL, respectively at 72 hours. As a reference, in porcine eyes receiving PBS vehicle control, dopaminergic derivatives in the anterior chamber were 0.86 ng/mL and in the vitreous 3.3 ng/mL.

### 2. In Vivo Pharmacokinetics of Etilevodopa

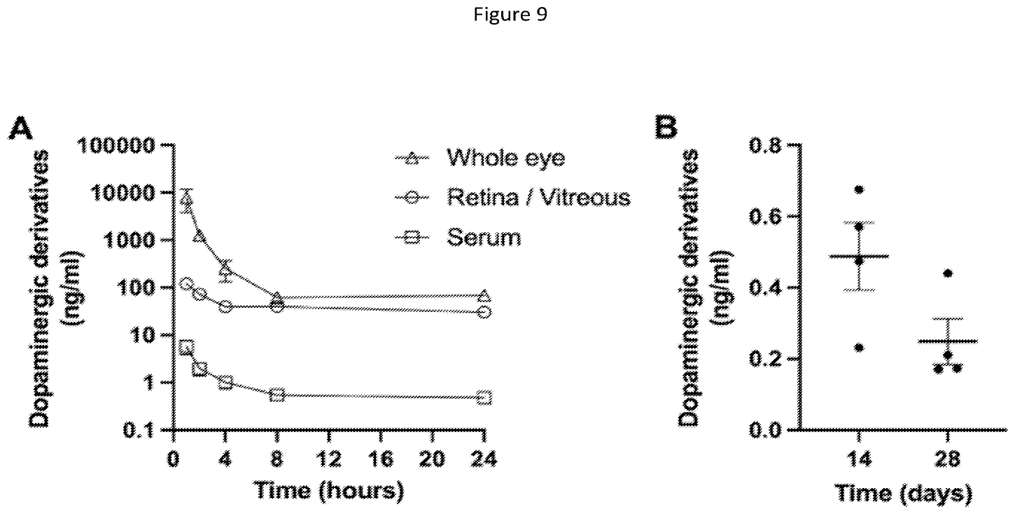

The in vivo 24-hour pharmacokinetic profile following a single topical treatment of etilevodopa 10% is shown in FIG. 9, panel A and the Table. A peak concentration of 7797.4 and 118.9 ng/mL were achieved in the whole eye and retina/vitreous at one hour after the treatment, respectively. This concentration declined to 62.1 ng/mL for the whole eye and 39.9 ng/mL for the retina/vitreous at eight hours after the treatment and maintained this level until 24 hours. In serum, a maximum concentration of 5.7 ng/mL was also achieved one hour after drug treatment, and the concentration fell to 0.48 ng/mL 24 hours after treatment. For the whole eye, etilevodopa has an area under the curve (AUC) of 4298.5 ng/mL, a half -life (T1/2) of 1.4 hours, and a mean resident time of 1.4 hours, whereas in the retina/vitreous, it has an AUC of 817.5 ng/mL, a T1/2 of 8.0 hours and a mean resident time of 11.7 hours. In the blood, it has an AUC of 11.2 ng/mL, a T1/2 of 3.4 hours and a mean resident time of 4.6 hours. Of note, the dopaminergic derivative concentration of retina/vitreous and serum in untreated Long

Evans rats were found to be 20.16 and 0.10 ng/mL, respectively.

TABLE
Noncompartmental Analysis of Dopaminergic Derivative
Concentrations in the Whole Rat Eye, Retina/Vitreous, and Blood
After a Single Topical Treatment of Etilevodopa 10% In Vivo

| Group | AUC_{0→∞} (ng/ml) | T_{1/2} (h) | Mean Resident Time (h) | R² |
|---|---|---|---|---|
| Whole eye | 4298.5 | 1.4 | 1.4 | 0.95 |
| Retina/ | 817.5 | 8.0 | 11.7 | 0.56 |
| Vitreous | | | | |
| Blood | 11.2 | 3.4 | 4.6 | 0.95 |

AUC, area under the curve;
T_{1/2,} half-life.

FIG. 9, Panel B shows the dopaminergic derivatives concentration in the rat serum at 14 and 28 days after once-daily etilevodopa administration. A concentration of 0.47 and 0.25 ng/mL was achieved at 14 and 28 days, respectively, which was comparable to serum concentration at 24 hours after a single treatment of etilevodopa (i.e., 0.48 ng/mL).

### 3. Clinical, Structural, and Functional Assessments

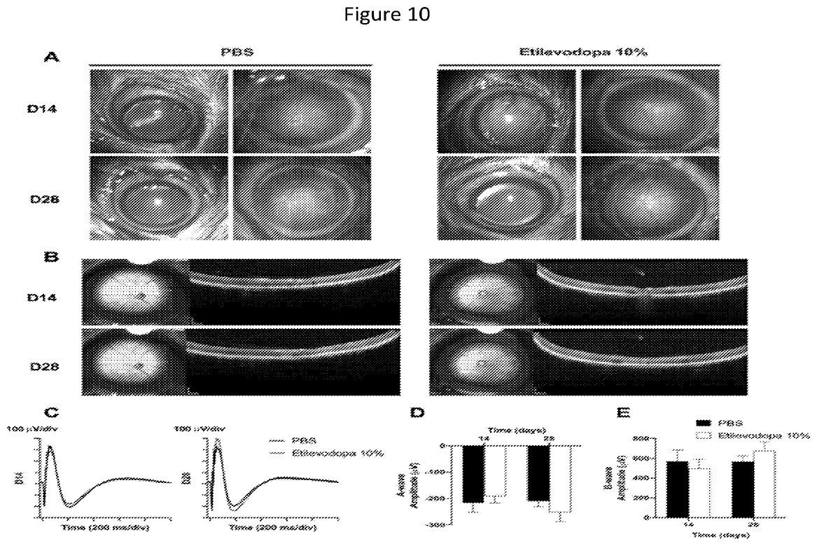

After once-daily treatment of etilevodopa 10% eye drop, the overall retina morphology and blood vessels remained unchanged (FIG. 10, Panel A) at 14 and 28 days. The cornea and lens remained clear throughout the duration of the study. No retinal detachment, edema or inflammation was observed in either the PBS control or the treatment groups. FIG. 10, Panel B compares the OCT images of the retina of rats receiving PBS control or etilevodopa 10% treatment. OCT did not demonstrate any qualitative structural changes in the retina at 14 and 28 days. Retinal function after the daily treatment of etilevodopa was measured using ERG recordings of the rod, cone, and mixed response. FIG. 10, Panel C shows the ERG waveforms of rat eyes treated with PBS control and etilevodopa at 14 and 28 days. The range of fluctuation of the ERG amplitudes of etilevodopa treated eyes was similar to control eyes. The comparison of both of A- and B-wave amplitude between the treated and control eyes are shown in 10, Panel D and FIG. 10, Panel E, and there was no statistically significant difference in A- or B-wave amplitudes at either 14 or 28 days.

### 4. Inflammation and Apoptosis

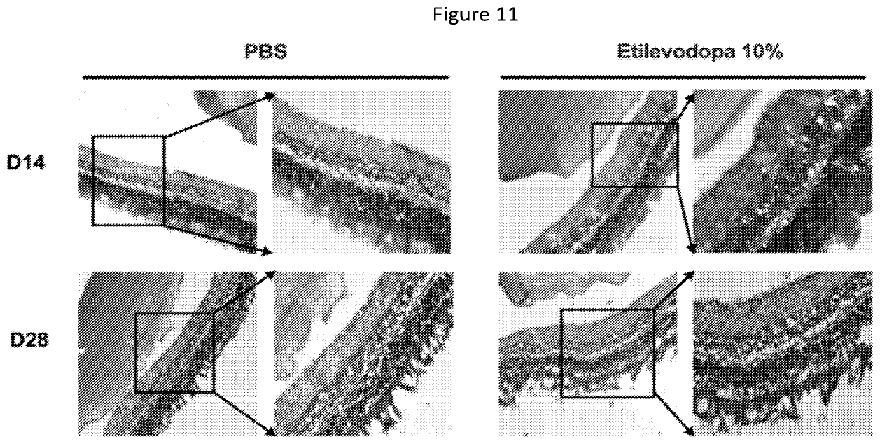

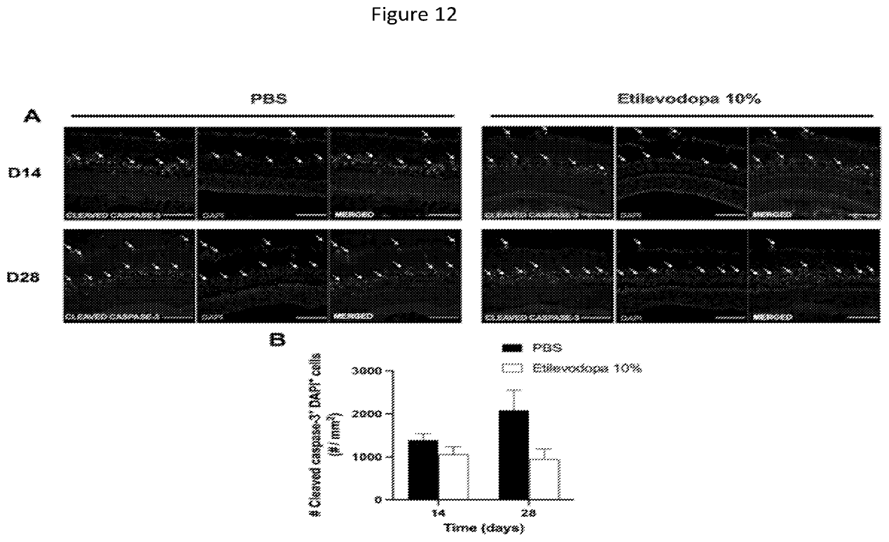

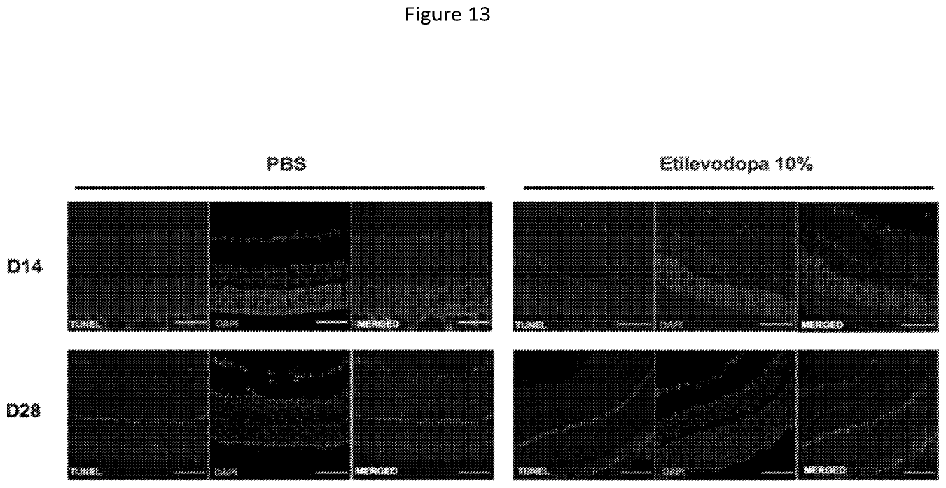

Histological observations including hematoxylin and eosin staining and immunohistochemistry assay (cleaved caspase-3 and TUNEL staining) were performed to further study the intraocular compatibility of etilevodopa in rat eyes. Hematoxylin and eosin staining (FIG. 11) shows that the retinal layer was intact at 14 and 28 days in both PBS control and etilevodopa treated rat eyes, with no infiltration of inflammatory cells, fibrosis or morphological changes. Next, apoptotic biomarkers, cleaved caspase-3 and TUNEL were labeled separately to further assess the effect of etilevodopa (FIGS. 12 and 13).^{31,32} FIG. 12 demonstrates the expression of cleaved caspase-3+ DAPI+ cells predominately located in inner nuclear layer in both PBS control and treatment groups at studied time points. There was no TUNEL+ DAPI+ cells observed in both PBS control and etilevodopa-treated groups at 14 and 28 days (FIG. 13). Of note, sections treated with DNase I to induce DNA stand breaks before TUNEL labeling procedure (according to manufacturer's instruction, Sigma-Aldrich) were studied as positive control (data not provided).

### D. Discussion

Animal models of myopia have consistently demonstrated a regulatory role of dopamine in the development of myopia. This occurs via dopaminergic amacrine cells that signal to the retinal pigment epithelium and choroid, leading ultimately to scleral remodeling and retinal neurogenesis. Given the potential role of dopamine in regulating axial elongation and the feasibility of topical administration for long-term treatment, dopaminergic agents have been explored as topical therapeutics. Unfortunately, dopaminergic agents often have poor solubility and ocular penetration limiting their usefulness as topical agents. The goal of this investigation was to determine whether improved ocular penetration would occur with dopaminergic prodrugs.

We assessed ocular penetration of dopamine and dopaminergic derivatives, including levodopa and etilevodopa. Levodopa is a dopamine precursor that is converted into dopamine via dopamine decarboxylase. It has recently been shown to be effective at slowing myopia progression in chicks after intravitreal administration and, to a lesser extent, after topical administration.^{25,29} Esterification has been an effective means of enhancing ocular penetration through improved solubility and penetration through the corneal stroma. Accordingly, dopaminergic ester prodrug, etilevodopa, was synthesized and ocular penetration was compared to levodopa. Etilevodopa is an ester prodrug of levodopa, and levodopa has demonstrated efficacy in preventing myopia progression in animal models when administered intravitreally, and to a lesser extent topically. It was therefore expected that etilevodopa would exhibit enhanced solubility and ocular penetration, making it a superior therapeutic myopia candidate.

We initially assessed ocular penetration in vitreous in enucleated porcine eyes (<30 hours old). Porcine eyes served as our ex vivo model as the globe size, cornea thickness, ratio of length of the cornea to eye-globe diameter, and histological structure of porcine eyes mimic the human eye. For direct comparison, all drugs were freshly prepared at 0.5% (the maximum concentration achievable with levodopa). An ELISA was used to quantify the presence of dopaminergic derivatives in the anterior chamber and vitreous. In vehicle-treated eyes, there was a higher quantity of dopaminergic derivatives in the vitreous compared to the anterior chamber. This is consistent with dopamine's role as a retinal neurotransmitter. Because dopamine diffuses from the retina, through the vitreous, and into the anterior chamber, vitreous concentrations are expected to be higher than the anterior chamber. Prior studies in humans have found a 3.5-fold higher concentration of vitreous dopamine (0.7 ng/mL) 33 as compared to anterior chamber dopamine (0.2 ng/mL).34 This study found a similar ratio of dopaminergic derivatives in control-treated eyes-0.86 ng/mL in the anterior chamber versus 3.3 ng/mL in the vitreous. The higher levels of background dopaminergic derivatives stem from quantification of not just dopamine but other dopaminergic derivatives (like 3,4-Dihydroxyphenylacetic acid (DOPAC)) and possible increased dopamine release from the retina after enucleation of porcine eyes. At 24 hours, all studied drugs at 0.5% showed increased dopaminergic derivatives into the vitreous (FIG. 7). We found that dopamine at 0.5% had the highest level of ocular penetration. Despite etilevodopa being an ester prodrug, it did not exhibit enhanced corneal penetration as compared to levodopa at a given concentration. However, because of its enhanced solubility, etilevodopa can readily achieve higher concentrations (10% etilevodopa vs. 0.5% levodopa) in standard physiological buffers facilitating clinical translation use. Interestingly, dopamine 0.5% exhibited the highest degree of ocular penetration, with a 10-fold higher intravitreal concentration as compared to etilevodopa 10% after topical administration in porcine eyes. There are several physiological and anatomical factors that can affect drug penetration through the cornea. The cornea consists of a lipophilic epithelial layer on the outside, a hydrophilic stromal layer in the middle and a much less lipophilic endothelial layer on the inside. The lipophilic layer can hinder the penetration of hydrophilic drugs, whereas the hydrophilic layer prevents the penetration of lipophilic drugs. Thus, drug molecules need to be amphiphilic in order to pass through the cornea.³⁴ Through this mechanism, ester prodrugs have consistently demonstrated enhanced corneal penetration and explain the improved anterior chamber and vitreous concentrations that were observed.^{30,34,35} However, unlike other ester prodrugs, we were unable to demonstrate improved corneal penetration after topical administration of etilevodopa as compared to levodopa. One possible explanation is that etilevodopa is rapidly hydrolyzed on the ocular surface before passing through the cornea. Another possibility is that etilevodopa and levodopa are primarily passing through the conjunctiva/sclera and not the cornea, making esterification less useful.

Similar to the ex vivo results, etilevodopa was able to enter the posterior segment in vivo (FIG. 9). After a single topical administration, a peak concentration of 7797.4, 118.9 and 5.7 ng/mL of etilevodopa was achieved in the whole rat eye, retina/vitreous and serum, respectively, at one hour (FIG. 9), and it reduced to 68.4, 30.3 and 0.48 ng/mL, respectively, at 24 hours. Increased dopamine levels are expected to increase dopamine receptor activity, which has been demonstrated to alter axial elongation.^{8,19,36} Although direct measurements of myopic eye growth after topical treatment of etilevodopa were not performed in this study, previous studies have shown that topical levodopa treatment slowed ocular growth and inhibited form deprivation myopia development in a dose-dependent manner in chicks.²⁵ Our work is the first to show ocular penetration with topical etilevodopa treatment in rats, and the finding supports the topical application of dopaminergic compounds as a potentially viable treatment approach for myopia. Additionally, based on our ex vivo data, etilevodopa 10% exhibited a higher vitreous penetration than levodopa 0.5% (etilevodopa: 36.2 ng/mL, levodopa: 11.9 ng/mL, P=0.0079). As a reference, Thomson et al. 25 demonstrated an EC50 for a reduction of axial elongation with topical levodopa of 0.05%. They further showed a stronger effect size with an intravitreal administration of levodopa (EC50 of 0.0008 mM in a 10 μL dose). Using a chick vitreous volume of 200 μL,³⁷ this EC50 is an intravitreal concentration of approximately 7.9 ng/mL. All tested compounds achieved intravitreal concentrations above this EC50 in our ex vivo model. However, the ex vivo porcine model significantly underestimates losses because of tear film turnover and conjunctival blood flow. Therefore, in an in vivo model, only dopamine 0.5% and to a lesser extend etilevodopa 10% would be expected to exceed the intravitreal EC50 concentration required to prevent axial elongation.

Dopamine is a major neurotransmitter in the vertebrate retina³⁸ and plays a significant role in regulation of center-surround antagonism.³⁹ It has both a synaptic and paracrine effect. Given its role in retinal signal processing, concern for retinal toxicity has been raised. Controversy still remains as to potential toxicity of levodopa due to increased oxidative stress on striatal neurons in Parkinson's disease. Although in vitro studies have demonstrated toxic effects,⁴⁰⁻⁴² most in vivo studies have failed to replicate such toxicity.⁴³⁻⁴⁴ In our study, four-week application of etilevodopa as a topical eye drop showed no evidence of direct toxicity. Structural testing with OCT did not demonstrate any change in retina morphology at 14 and 28 days, whereas functional testing with ERG failed to identify reductions in A- or B-wave amplitude (FIG. 10). Histology and immunohistochemistry demonstrated that etilevodopa did not change retinal architecture or induce any inflammation or cell death (apoptosis) compared to PBS control (FIGS. 11, 12 and 13). Together, these initial safety studies indicate that topical etilevodopa may have a safety profile that supports its use as a long-term therapeutic treatment for myopia.

Delivery of dopaminergic agents via peribulbar, intravitreal, or intraperitoneal injections through the wall of the eye can lead to several complications such as ocular irritation, inflammation, conjunctivitis, internal ocular bleeding, and formation of cataracts, especially with repeated injections. These complications are not associated with topical eye drops. It is known that topical drug treatment has potential off-target effects due to systemic distribution.²⁹ However, in this study, the serum concentration at 14 and 28 days after once-daily etilevodopa treatment were comparable to the concentration after a single treatment at 24 hours (FIG. 9), suggesting that the systemic distribution for etilevodopa is limited, and it does not accumulate in the body after repeated daily treatment, which may minimize any nonocular off-target side effects. Further supporting these findings, we observed no obvious changes in rat behavior during the 28-day study, including activity levels, aggression, feeding pattern, and weight gain. However, additional testing is required to confirm the lack of effect on behavioral changes.

### E. Conclusion

We have successfully demonstrated that topical administration of etilevodopa can penetrate the eye and enter the posterior segment. Topical etilevodopa did not exhibit enhanced ocular penetration as compared to levodopa but, because of its enhanced solubility, was able to be applied in a more concentrated form and thus result in greater intravitreal concentrations. Surprisingly, dopamine exhibited the greatest ocular penetration after topical administration. Considering the feasibility of using topical administration, these findings highlight indicate that dopamine and etilevodopa find use as a therapeutic treatment for myopia.

### F. References

1. Dolgin E. The myopia boom. Nature. 2015; 519(7543): 276-278.
2. Holden B A, Fricke T R, Wilson D A, et al. . . . Global prevalence of myopia and high myopia and temporal trends from 2000 through 2050. Ophthalmology. 2016; 123: 1036-1042.
3. Ikuno Y. Overview of the complications of high myopia. Retina. 2017; 37: 2347-2351.
4. Wang W-Y, Chen C, Chang J, et al. . . . Pharmacotherapeutic candidates for myopia: a review. Biomed Pharmacother. 2021; 133: 111092.
5. Stone R A, Lin T, Laties A M, Iuvone P M. . . . Retinal dopamine and form-deprivation myopia. Proc Natl Acad Sci USA. 1989; 86: 704-706.
6. Rohrer B, Spira A W, Stell W K. . . . Apomorphine blocks form-deprivation myopia in chickens by a dopamine D2-receptor mechanism acting in retina or pigmented epithelium. Vis Neurosci. 1993; 10: 447-453.
7. Mao J, Liu S, Qin W, Li F, Wu X, Tan Q. . . . Levodopa inhibits the development of form-deprivation myopia in guinea pigs. Optom Vis Sci. 2010; 87: 53-60.
8. Nickla D L, Totonelly K, Dhillon B. . . . Dopaminergic agonists that result in ocular growth inhibition also elicit transient increases in choroidal thickness in chicks. Exp Eye Res. 2010; 91: 715-720.
9. Nickla DL, Lee L, Totonelly K. . . . Nitric oxide synthase inhibitors prevent the growth-inhibiting effects of quinpirole. Optom Vis Sci. 2013; 90: 1167-1175.
10. Gao Q, Liu Q, Ma P, Zhong X, Wu J, Ge J. . . . Effects of direct intravitreal dopamine injections on the development of lid-suture induced myopia in rabbits. Graefes Arch Clin Exp Ophthalmol. 2006; 244: 1329-1335.
11. Lin Z, Chen X, Ge J, et al. Effects of direct intravitreal dopamine injection on sclera and retina in form-deprived myopic rabbits. J Ocul Pharmacol Ther. 2008; 24: 543-550.
12. Mao J, Liu S, Qin W, Xiang Q, Wu X. . . . Exogenous levodopa increases the neuro retinal dopamine of guinea pig myopic eyes in vitro. Eye Sci. 2011; 26: 211-216.
13. Dong F, Zhi Z, Pan M, et al. . . . Inhibition of experimental myopia by a dopamine agonist: different effectiveness between form deprivation and hyperopic defocus in guinea pigs. Mol Vis. 2011; 17: 2824-2834.
14. Huang F, Yan T, Shi F, et al. . . . Activation of dopamine D2 receptor is critical for the development of form-deprivation myopia in the C57BL/6 mouse. Invest Ophthalmol Vis Sci. 2014; 55: 5537-5544.
15. Huang F, Zhang L, Wang Q, et al. . . . Dopamine D1 receptors contribute critically to the apomorphine-induced inhibition of form-deprivation myopia in mice. Invest Ophthalmol Vis Sci. 2018; 59: 2623-2634.
16. Yan T, Xiong W, Huang F, et al. . . . Daily injection but not continuous infusion of apomorphine inhibits form-deprivation myopia in mice. Invest Ophthalmol Vis Sci. 2015; 56: 2475-2485.
17. Ward A H, Siegwart J T, Frost M R, Norton T T. . . . Intravitreally-administered dopamine D2-like (and D4), but not D1-like, receptor agonists reduce form-deprivation myopia in tree shrews. Vis Neurosci. 2017; 34: E003.
18. Iuvone P M, Tigges M, Stone R A, Lambert S, Laties A M. . . . Effects of apomorphine, a dopamine receptor agonist, on ocular refraction and axial elongation in a primate model of myopia. Invest Ophthalmol Vis Sci. 1991; 32: 1674-1677.
19. McCarthy C S, Megaw P, Devadas M, Morgan IG. . . . Dopaminergic agents affect the ability of brief periods of normal vision to prevent form-deprivation myopia. Exp Eye Res. 2007; 84: 100-107.
20. Zhou X, Xiong W, Huang F, Ji Yang, Qu J. . . . C57BL/6 mouse eyes treated by dopamine D1 receptor agonist and antagonist during form deprivation: an opposite effect on axial length and refractive development. Invest Ophthalmol Vis Sci. 2014; 55: 3038-3038.
21. Ashby R, McCarthy C S, Maleszka R, Megaw P, Morgan I G. . . . A muscarinic cholinergic antagonist and a dopamine agonist rapidly increase ZENK mRNA expression in the form-deprived chicken retina. Exp Eye Res. 2007; 85: 15-22.
22. Schmid K L, Wildsoet C F. . . . Inhibitory effects of apomorphine and atropine and their combination on myopia in chicks. Optom Vis Sci. 2004; 81: 137-147.
23. Jiang L, Long K, Schaeffel F, et al. . . . Effects of dopaminergic agents on progression of naturally occurring myopia in albino guinea pigs (Cavia porcellus). Invest Ophthalmol Vis Sci. 2014; 55: 7508-7519.
24. Yeung L, Hung P T, Lin L L, Yang C H, Chiou G C. . . . Effects of dopamine antagonists in human eye accommodation. J Ocul Pharmacol Ther. 2001; 17: 11-17.
25. Thomson K, Karouta C, Morgan I, Kelly T, Ashby R. . . . Effectiveness and safety of topical levodopa in a chick model of myopia. Sci Rep. 2019; 9(1): 18345.
26. Kothari M, Bhat D, Khadse N, Jain R, Rathod V, Am P. . . . Changes in pattern electroretinogram after application of 0.01% atropine eye drops. Indian J Ophthalmol. 2019; 67: 309-310.
27. Khanal S, Rathod S N, Phillips J R. . . . The acute effect of atropine eye drops on the human full-field electroretinogram. Doc Ophthalmol. 2021; 142: 315-328.
28. COMET Group. Myopia stabilization and associated factors among participants in the Correction of Myopia Evaluation Trial (COMET). Invest Ophthalmol Vis Sci. 2013; 54: 7871-7884.
29. Thomson K, Morgan I, Karouta C, Ashby R. . . . Levodopa inhibits the development of lens-induced myopia in chicks. Sci Rep. 2020; 10: 13242.
30. Taskar P, Tatke A, Majumdar S. . . . Advances in the use of prodrugs for drug delivery to the eye. Expert Opin Drug Deliv. 2017; 14: 49-63.
31. Slee E A, Adrain C, Martin S J. . . . Executioner caspase-3, -6, and -7 perform distinct, non-redundant roles during the demolition phase of apoptosis. J Biol Chem. 2001; 276: 7320-7326.
32. Kyrylkova K, Kyryachenko S, Leid M, Kioussi C. . . . Detection of apoptosis by TUNEL assay. Methods Mol Biol. 2012; 887: 41-47.
33. Hendrick A, Smith J, Stelton C, et al. . . . Dopamine metabolite levels in the vitreous of diabetic and non-diabetic humans. Exp Eye Res, 2020; 195: 108040, doi:10.1016/j.exer.2020.108040.
34. Cooper R L, Constable I J, Davidson L. . . . Catecholamines in aqueous humour of glaucoma patients. Aust J Ophthalmol. 1984; 12: 345-349.
35. Lee V H L. Mechanisms and facilitation of corneal drug penetration. Journal of Controlled Release. 1990; 11: 79-90.
36. Ibrahim M M, Maria D N, Wang X, Simpson R N, Hollingsworth T J, Jablonski M M. . . . Enhanced corneal penetration of a poorly permeable drug using bioadhesive multiple microemulsion technology, Pharmaceutics. 2020; 12: 704, doi:10.3390/pharmaceutics12080704.
37. Schaeffel F, Bartmann M, Hagel G, Zrenner E. . . . Studies on the role of the retinal dopamine/melatonin system in experimental refractive errors in chickens. Vision Res. 1995; 35: 1247-1264.
38. Diether S, Schaeffel F, Lambrou G N, Fritsch C, Trendelenburg A-U. . . . Effects of intravitreally and intraperitonally injected atropine on two types of experimental myopia in chicken. Exp Eye Res. 2007; 84: 266-274.
39. Djamgoz M B, Hankins M W, Hirano J, Archer S N. . . . Neurobiology of retinal dopamine in relation to degenerative states of the tissue. Vision Res. 1997; 37: 3509-3529.
40. Dowling J E. Dopamine: a retinal neuromodulator? Trends Neurosci. 1986; 9: 236-240.
41. Mosharov EV, Larsen KE, Kanter E, et al. . . . Interplay between cytosolic dopamine, calcium, and alpha-synuclein causes selective death of substantia nigra neurons. Neuron. 2009; 62: 218-229.
42. Mytilineou C, Han S K, Cohen G. . . . Toxic and protective effects of L-dopa on mesencephalic cell cultures. J Neurochem. 1993; 61: 1470-1478.
43. Rajput A H, M E Fenton, Birdi S, Macaulay R. Is levodopa toxic to human substantia nigra? Mov Disord. 1997; 12: 634-638.
44. Schapira A H V. The clinical relevance of levodopa toxicity in the treatment of Parkinson's disease. Mov Disord. 2008; 23(Suppl 3): S515-S520.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims. The scope of the present invention, therefore, is not intended to be limited to the exemplary embodiments shown and described herein. Rather, the scope and spirit of present invention is embodied by the appended claims. In the claims, 35 U.S.C. §112(f) or 35 U.S.C. §112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. §112(6) is not invoked.

1. An ophthalmic pharmaceutical composition comprising:
a dopamine agonist prodrug; and
a pharmaceutically acceptable delivery vehicle.

2. The ophthalmic pharmaceutical composition according to claim 1, wherein the dopamine agonist product is converted to a dopamine agonist upon contact with an esterase.

3. The ophthalmic pharmaceutical composition according to claim 1, wherein the esterase is an ocular surface esterase.

4. The ophthalmic pharmaceutical composition according to claim 1, wherein the composition further comprises a dopamine agonist inactivating agent.

5. The ophthalmic pharmaceutical composition according to claim 1, wherein the dopamine agonist prodrug is a prodrug of a selective dopamine agonist.

6. The ophthalmic pharmaceutical composition according to claim 5, wherein selective dopamine agonist is a selective D1 agonist.

7. The ophthalmic pharmaceutical composition according to claim 5, wherein the selective dopamine agonist is a selective D2 agonist.

8. The ophthalmic pharmaceutical composition according to claim 1, wherein the dopamine agonist is a non-selective dopamine agonist.

9. The ophthalmic pharmaceutical composition according to claim 1, wherein the composition further comprises dopamine antagonist or prodrug thereof.

10. The ophthalmic pharmaceutical composition according to claim 1, wherein the composition further comprises a dopamine reuptake inhibitor.

11. The ophthalmic pharmaceutical composition according to claim 1, wherein the composition further comprises a vesicular monoamine transport inhibitor.

12. The ophthalmic pharmaceutical composition according to claim 1, wherein the composition has a form selected from the group consisting of a solution, a suspension and an ointment.

13. A method comprising administering a composition according to claim 1 to an eye.

14. The method according to claim 13, wherein the composition is topically administered to a surface of the eye.

15. The method according to claim 13, wherein the composition is intravitreally administered to the eye.

16. The method according to claim 1, wherein the method is a method of inhibiting axial elongation.

17. The method according to claim 1, wherein the method is a method of treating a subject for myopia.

18. A method of treating a subject for myopia, the method comprising repeatedly administering a composition according to claim 1 to an eye of the subject to treat the subject for myopia.

19. The method according to claim 18, wherein repeatedly administering comprises administering once per day.

20. The method according to claim 18, wherein the subject is a human.
